SEQUENCE LISTING (101 sequences)

SEQ ID NO: 1 - protein, 136 aa, Artificial Sequence
  Canine IL-31 protein
SHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 2 - protein, 331 aa, Artificial Sequence
  canine immunoglobulin gamma heavy chain A
ASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 3-5 - skipped (3 entries)

SEQ ID NO: 6 - protein, 8 aa, Artificial Sequence
  monoclonal antibody RD4.D2 - HC_CDR1
GYTFTDYS

SEQ ID NO: 7 - protein, 8 aa, Artificial Sequence
  monoclonal antibody RD4.D2 - HC_CDR2
INTETGKT

SEQ ID NO: 8 - protein, 7 aa, Artificial Sequence
  monoclonal antibody RD4.D2  - HC_CDR3
ASGRPDY

SEQ ID NO: 9 - protein, 10 aa, Artificial Sequence
  monoclonal antibody RD4.D2 - LC_CDR1
ESVEYYGTSL

SEQ ID NO: 10 - protein, 3 aa, Artificial Sequence
  monoclonal antibody RD4.D2 - LC_CDR2
AAS

SEQ ID NO: 11 - protein, 9 aa, Artificial Sequence
  monoclonal antibody RD4.D2 - LC_CDR3
QQSRKVPYT

SEQ ID NO: 12 - protein, 114 aa, Artificial Sequence
  murine monoclonal antibody RA4.D2 - VH
QIQLVQSGPELKKPGETVKISCKASGYTFTDYSMHWVKQAPGKGLRWMGWINTETGKTIHAEDFKGRVAFSLETSASTAYLQINNLKNEDTATYFCASGRPDYWGQGTTLTVSS

SEQ ID NO: 13 - protein, 120 aa, Artificial Sequence
  murine monoclonal antibody RA4.D2 - VL
DIVLSQSPASLAVSLGQRATISCRASESVEYYGTSLMQWYQQKPGQPPKLLIYAASGVESGVPARFSGSGSGTDFSLIIHPVEEDDIAMYFCQQSRKVPYTFGGGTKLEIKRADAAPTVS

SEQ ID NO: 14 - protein, 342 aa, Artificial Sequence
  murine monoclonal antibody RA4.D2 - Heavy-chain variable region (VH)
CAGATCCAGTTGGTGCAGTCTGGACCTGAGCTGAAGAAGCCAGGAGAGACAGTCAAGATCTCCTGCAAGGCTTCTGGTTATACCTTCACAGACTATTCAATGCACTGGGTGAAGCAGGCTCCAGGAAAGGGTTTAAGGTGGATGGGCTGGATAAACACTGAGACTGGTAAGACAATACACGCAGAAGACTTCAAGGGACGGGTTGCCTTCTCTTTGGAAACCTCTGCCAGCACTGCCTATTTGCAGATCAACAACCTCAAAAATGAGGACACGGCTACATATTTCTGTGCTAGCGGGCGGCCTGACTACTGGGGCCAAGGCACCACTCTCACAGTCTCCTCA

SEQ ID NO: 15 - protein, 336 aa, Artificial Sequence
  murine monoclonal antibody RA4.D2 - Light-chain variable region (VL); (kappa light chain)
GACATTGTGCTCTCCCAATCTCCAGCTTCTTTGGCTGTGTCTCTAGGGCAGAGAGCCACCATCTCCTGCAGAGCCAGTGAAAGTGTTGAATATTATGGCACAAGTTTGATGCAGTGGTACCAACAGAAGCCAGGACAGCCACCCAAACTCCTCATCTATGCTGCATCCGGCGTAGAATCTGGGGTCCCTGCCAGGTTTAGTGGCAGTGGGTCTGGGACAGACTTCAGCCTCATCATCCATCCTGTGGAGGAGGATGATATTGCAATGTATTTCTGTCAGCAAAGTAGGAAGGTTCCGTACACGTTCGGAGGGGGGACCAAACTGGAAATAAAACGG

SEQ ID NO: 16 - protein, 445 aa, Artificial Sequence
  chimeric RA4.D2 antibody - heavy chain (HC)
QIQLVQSGPELKKPGETVKISCKASGYTFTDYSMHWVKQAPGKGLRWMGWINTETGKTIHAEDFKGRVAFSLETSASTAYLQINNLKNEDTATYFCASGRPDYWGQGTTLTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 17 - protein, 221 aa, Artificial Sequence
  chimeric RA4.D2 antibody - light chain (LC)
DIVLSQSPASLAVSLGQRATISCRASESVEYYGTSLMQWYQQKPGQPPKLLIYAASGVESGVPARFSGSGSGTDFSLIIHPVEEDDIAMYFCQQSRKVPYTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 18 - protein, 25 aa, Artificial Sequence
  Caninized VH Framework Region (FR) - VH1 FR1
QVQLQESGPGLVKPSQTLSLTCTVS

SEQ ID NO: 19 - protein, 14 aa, Artificial Sequence
  Caninized VH Framework Region (FR) - VH1 FR2
WVRQRTGRGLEWMG

SEQ ID NO: 20 - protein, 32 aa, Artificial Sequence
  Caninized VH Framework Region (FR) - VH1 FR3
RLSITRDTAKSQVSLQMSSMTTEDTATYYCAR

SEQ ID NO: 21 - protein, 11 aa, Artificial Sequence
  Caninized VH Framework Region (FR) - VH1 FR4
WGQGTSVTVSS

SEQ ID NO: 22 - protein, 25 aa, Artificial Sequence
  Caninized VH Framework Region (FR) - VH2 FR1
QVQLQESGPGLVKPSQTLSLTCTVS

SEQ ID NO: 23 - protein, 14 aa, Artificial Sequence
  Caninized VH Framework Region (FR) - VH2 FR2
WVRQRPGRGLEWMG

SEQ ID NO: 24 - protein, 32 aa, Artificial Sequence; identical to SEQ ID NO: 20
  Caninized VH Framework Region (FR) - VH2 FR3

SEQ ID NO: 25 - protein, 11 aa, Artificial Sequence
  Caninized VH Framework Region (FR) - VH2 FR4
WGQGTLVTVSS

SEQ ID NO: 26 - protein, 25 aa, Artificial Sequence
  Caninized VH Framework Region (FR) - VH3 FR1
QVQLQESGPGLVKPSQTLSLTCTVS

SEQ ID NO: 27 - protein, 14 aa, Artificial Sequence
  Caninized VH Framework Region (FR) - VH3 FR3
WVRQRPGRGLEWMG

SEQ ID NO: 28 - protein, 32 aa, Artificial Sequence
  Caninized VH Framework Region (FR) - VH3 FR3
RISITRDTAKSQVSLQLSSMTTEDTATYYCAR

SEQ ID NO: 29 - protein, 11 aa, Artificial Sequence
  Caninized VH Framework Region (FR) - VH3 FR4
WGQGTLVTVSS

SEQ ID NO: 30 - protein, 25 aa, Artificial Sequence
  Caninized VH Framework Region (FR) - VH4 FR1
EVTLQESGPGLVKPSQTLSLTCTVS

SEQ ID NO: 31 - protein, 14 aa, Artificial Sequence
  Caninized VH Framework Region (FR) - VH4 FR2
WVRQRPGRGLEWMG

SEQ ID NO: 32 - protein, 32 aa, Artificial Sequence
  Caninized VH Framework Region (FR) - VH4 FR3
RISITRDTAKNQVSLQLSSMTTEDTATYYCAR

SEQ ID NO: 33 - protein, 11 aa, Artificial Sequence
  Caninized VH Framework Region (FR) - VH4 FR4
WGQGTLVTVSS

SEQ ID NO: 34 - protein, 25 aa, Artificial Sequence
  Caninized VH Framework Region (FR) - VH5 FR1
EVTLQESGPGLVKPSQTLSLTCVVS

SEQ ID NO: 35 - protein, 14 aa, Artificial Sequence
  Caninized VH Framework Region (FR) - VH5 FR2
WVRQRPGRGLEWMG

SEQ ID NO: 36 - protein, 32 aa, Artificial Sequence
  Caninized VH Framework Region (FR) - VH5 FR3
RISITRDTAKNQVSLQLSSMTTEDTAVYYCAR

SEQ ID NO: 37 - protein, 11 aa, Artificial Sequence
  Caninized VH Framework Region (FR) - VH5 FR4
WGQGTLVTVSS

SEQ ID NO: 38 - protein, 23 aa, Artificial Sequence
  Caninized VL Framework Region (FR) - VL1 FR1
DIQMTQSPASLSLSQEEKVTITC

SEQ ID NO: 39 - protein, 15 aa, Artificial Sequence
  Caninized VL Framework Region (FR) - VL1 FR2
WYQQKPGQAPKLLIY

SEQ ID NO: 40 - protein, 32 aa, Artificial Sequence
  Caninized VL Framework Region (FR) - VL1 FR3
GVPSRFSGSGSGTDYSFTISSLESEDVASYFC

SEQ ID NO: 41 - protein, 10 aa, Artificial Sequence
  Caninized VL Framework Region (FR) - VL1 FR4
FGAGTKVELK

SEQ ID NO: 42 - protein, 23 aa, Artificial Sequence
  Caninized VL Framework Region (FR) - VL2 FR1
DIQMTQSPASLSLSQEEKVTITC

SEQ ID NO: 43 - protein, 15 aa, Artificial Sequence
  Caninized VL Framework Region (FR) - VL2 FR2
WYQQKPGQAPKLLIY

SEQ ID NO: 44 - protein, 32 aa, Artificial Sequence
  Caninized VL Framework Region (FR) - VL2 FR3
GVPSRFSGSGSGTDYSFTISSLEPEDVASYFC

SEQ ID NO: 45 - protein, 10 aa, Artificial Sequence
  Caninized VL Framework Region (FR) - VL2 FR4
FGAGTKVELK

SEQ ID NO: 46 - protein, 23 aa, Artificial Sequence
  Caninized VL Framework Region (FR) - VL3 FR1
EIVMTQSPASLSLSQEEKVTITC

SEQ ID NO: 47 - protein, 15 aa, Artificial Sequence
  Caninized VL Framework Region (FR) - VL3 FR2
WYQQKPGQAPKLLIY

SEQ ID NO: 48 - protein, 32 aa, Artificial Sequence; identical to SEQ ID NO: 44
  Caninized VL Framework Region (FR) - VL3 FR3

SEQ ID NO: 49 - protein, 10 aa, Artificial Sequence
  Caninized VL Framework Region (FR) - VL3 FR4
FGAGTKVELK

SEQ ID NO: 50 - protein, 23 aa, Artificial Sequence
  Caninized VL Framework Region (FR) - VL4 FR1
EIVMTQSPASLSLSQEEKVTITC

SEQ ID NO: 51 - protein, 15 aa, Artificial Sequence
  Caninized VL Framework Region (FR) - VL4 FR2
WYQQKPGQAPKLLIY

SEQ ID NO: 52 - protein, 32 aa, Artificial Sequence
  Caninized VL Framework Region (FR) - VL4 FR3
GVPSRFSGSGSGTDYSFTISSLEPEDVAVYFC

SEQ ID NO: 53 - protein, 10 aa, Artificial Sequence
  Caninized VL Framework Region (FR) - VL4 FR4
FGAGTKVELK

SEQ ID NO: 54 - protein, 25 aa, Artificial Sequence
  Felinised VH Framework Region (FR) - FR1
QVQLMESGADLVQPSESLRLTCVAS

SEQ ID NO: 55 - protein, 14 aa, Artificial Sequence
  Felinised VH Framework Region (FR) - FR2
WVRQAPGKGLEWMG

SEQ ID NO: 56 - protein, 32 aa, Artificial Sequence
  Felinised VH Framework Region (FR) - FR3
RLTITRDTSKNTVFLQMHSLQSEDTATYYCAR

SEQ ID NO: 57 - protein, 11 aa, Artificial Sequence
  Felinised VH Framework Region (FR) - FR4
WGQGTTVTVSA

SEQ ID NO: 58 - protein, 23 aa, Artificial Sequence
  Felinised VL Framework Region (FR) - FR1
DIEMTQSPLSLSATPGETVSISC

SEQ ID NO: 59 - protein, 15 aa, Artificial Sequence
  Felinised VL Framework Region (FR) - FR2
WYLQKPGRSPRLLIY

SEQ ID NO: 60 - protein, 32 aa, Artificial Sequence
  Felinised VL Framework Region (FR) - FR3
GVPDRFSGSGSGTDFTLKISRVQTEDVGVYFC

SEQ ID NO: 61 - protein, 10 aa, Artificial Sequence
  Felinised VL Framework Region (FR) - FR4
FGQGTKLELK

SEQ ID NO: 62 - protein, 136 aa, Artificial Sequence
  Canine IL-31 Mutant 1
SHMAPTHQLPPSDVRKIILELQPLSRGLLEDYQKKETGVPESNRTLLLCLTSDSQPPRLNSSAILPYFRAIRPLSAANIAAKIAEQLDKLKFQHEPETEISVPADTFECKSFILTILQQFSACLESVFKSLNSGPQ

SEQ ID NO: 63 - protein, 136 aa, Artificial Sequence
  Canine IL-31 Mutant 2
SHMAPTHQLPPSAVAKIILALQPLSAGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 64 - protein, 136 aa, Felis catus
SHMAPAHRLQPSDVRKIILELRPMSKGLLQDYLKKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 65 - protein, 140 aa, Artificial Sequence
  Canine/Human chIL-31_c123_h4
SHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETNISVPTDTHECKRFILTISQQFSECMDLALKSLTSGAQQATT

SEQ ID NO: 66 - protein, 140 aa, Artificial Sequence
  Canine/Human chIL-31_c12_h34
SHMAPTHQLPPSDVRKIILELQPLSRGLLEDYQKKETGVPESNRTLLLCLTSDSQPPRLNSSAILPYFRAIRPLSDKSVIDEIIEHLDKLIFQDAPETNISVPTDTHECKRFILTISQQFSECMDLALKSLTSGAQQATT

SEQ ID NO: 67 - protein, 141 aa, Artificial Sequence
  Canine/Human chIL-31_c1_h234
SHMAPTHQLPPSDVRKIILELQPLSRGLLEDYQKKETGVPESNRTLLLCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 68 - protein, 49 aa, Artificial Sequence
  Canine IL-31_N-term
SHMAPTHQLPPSDVRKIILELQPLSRGLLEDYQKKETGVPESNRTLLLC

SEQ ID NO: 69 - protein, 20 aa, Artificial Sequence
  Canine IL-31_N-term_20_1
SHMAPTHQLPPSDVRKIILE

SEQ ID NO: 70 - protein, 20 aa, Artificial Sequence
  Canine IL-31_N-term_20_2
PSDVRKIILELQPLSRGLLE

SEQ ID NO: 71 - protein, 20 aa, Artificial Sequence
  Canine IL-31_N-term_20_3
LQPLSRGLLEDYQKKETGVP

SEQ ID NO: 72 - protein, 20 aa, Artificial Sequence
  Canine IL-31_N-term_20_4
DYQKKETGVPESNRTLLLCL

SEQ ID NO: 73 - protein, 165 aa, Artificial Sequence
  Canine IL-31
MLSHTGPSRFALFLLCSMETLLSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQHHHHHH

SEQ ID NO: 74 - protein, 165 aa, Artificial Sequence
  Canine IL-31_4xMUT_combo
MLSHTGPSRFALFLLCSMETLLSSHMAPTHQLPPSDVRKIILELQPLSRGLAEDAAKKETGVAESNRTLLLCLTSDSQPPRLNSSAILPYFRAIRPLSDKNIIDKIIEQLDKLKFQHEPETEISVPADTFECKSFILTILQQFSACLESVFKSLNSGPQHHHHHH

SEQ ID NO: 75 - protein, 165 aa, Artificial Sequence
  Canine IL-31_L29A
MLSHTGPSRFALFLLCSMETLLSSHMAPTHQLPPSDVRKIILELQPLSRGLAEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQHHHHHH

SEQ ID NO: 76 - protein, 165 aa, Artificial Sequence
  Canine IL-31_Y32A
MLSHTGPSRFALFLLCSMETLLSSHMAPTHQLPPSDVRKIILELQPLSRGLLEDAQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQHHHHHH

SEQ ID NO: 77 - protein, 165 aa, Artificial Sequence
  Canine IL-31_Q33A
MLSHTGPSRFALFLLCSMETLLSSHMAPTHQLPPSDVRKIILELQPLSRGLLEDYAKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQHHHHHH

SEQ ID NO: 78 - protein, 165 aa, Artificial Sequence
  Canine IL-31_P40A
MLSHTGPSRFALFLLCSMETLLSSHMAPTHQLPPSDVRKIILELQPLSRGLLEDYQKKETGVAESNRTLLLCLTSDSQPPRLNSSAILPYFRAIRPLSDKNIIDKIIEQLDKLKFQHEPETEISVPADTFECKSFILTILQQFSACLESVFKSLNSGPQHHHHHH

SEQ ID NO: 79 - protein, 26 aa, Artificial Sequence
  alanine-scanning peptide library of the region encompassing amino acid residues 18-42 of SEQ ID NO:1
IILELQPLSRGLLEDYQKKETGVPES

SEQ ID NO: 80 - protein, 110 aa, Artificial Sequence
  Canine kappa Light Chain Constant Region
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 81 - protein, 25 aa, Artificial Sequence
  Caninized VH Framework Region (FR) Sequences - VH6 - FR1
EVQLVQSGPEVKKPGESVKVSCKTS

SEQ ID NO: 82 - protein, 14 aa, Artificial Sequence
  Caninized VH Framework Region (FR) Sequences - VH6 - FR2
WVRQAPGKGLRWMG

SEQ ID NO: 83 - protein, 30 aa, Artificial Sequence
  Caninized VH Framework Region (FR) Sequences - VH6 - FR3
RVTLTADTSTSTAYMQLSSLRAEDIAVYFC

SEQ ID NO: 84 - protein, 11 aa, Artificial Sequence
  Caninized VH Framework Region (FR) Sequences - VH6 - FR4
WGQGTTLTVSS

SEQ ID NO: 85 - protein, 25 aa, Artificial Sequence
  Caninized VH Framework Region (FR) Sequences - VH7 - FR1
QVQLVQSGAEVKKPGESVKVSCKTS

SEQ ID NO: 86 - protein, 14 aa, Artificial Sequence
  Caninized VH Framework Region (FR) Sequences - VH7 - FR2
WVRQAPGKGLDWMG

SEQ ID NO: 87 - protein, 30 aa, Artificial Sequence
  Caninized VH Framework Region (FR) Sequences - VH7 - FR3
RVTLTADTSTSTAYMELSSLRAGDIAVYFC

SEQ ID NO: 88 - protein, 11 aa, Artificial Sequence
  Caninized VH Framework Region (FR) Sequences - VH7 - FR4
WGQGTTLTVSS

SEQ ID NO: 89 - protein, 23 aa, Artificial Sequence
  Caninized VH Framework Region (FR) Sequences - VL5 - FR1
EIVLTQSPASLSLSQGEKVTITC

SEQ ID NO: 90 - protein, 15 aa, Artificial Sequence
  Caninized VH Framework Region (FR) Sequences - VL5 - FR2
WYQQKPGQAPKLLIY

SEQ ID NO: 91 - protein, 32 aa, Artificial Sequence
  Caninized VH Framework Region (FR) Sequences - VL5 - FR3
GVPSRFSGSGSGTDFSFTISSLEPEDVAVYFC

SEQ ID NO: 92 - protein, 10 aa, Artificial Sequence
  Caninized VH Framework Region (FR) Sequences - VL5 - FR4
FGGGTKLEIK

SEQ ID NO: 93 - protein, 5 aa, Artificial Sequence
  RA4.D2 - HC_CDR1
DYSMH

SEQ ID NO: 94 - protein, 17 aa, Artificial Sequence
  RA4.D2 - HC_CDR2
WINTETGKTIHAEDFKG

SEQ ID NO: 95 - protein, 5 aa, Artificial Sequence
  RA4.D2 - HC_CDR3
GRPDY

SEQ ID NO: 96 - protein, 15 aa, Artificial Sequence
  RA4.D2 - LC_CDR1
RASESVEYYGTSLMQ

SEQ ID NO: 97 - protein, 7 aa, Artificial Sequence
  RA4.D2 - LC_CDR2
AASGVES

SEQ ID NO: 98 - protein, 9 aa, Artificial Sequence
  RA4.D2 - LC_CDR3
QQSRKVPYT

SEQ ID NO: 99 - protein, 114 aa, Artificial Sequence
  RA4 Caninised Heavy Chain - VHC5
EVQLVQSGPEVKKPGESVKVSCKTSGYTFTDYSMHWVRQAPGKGLRWMGWINTETGKTIHAEDFKGRVTLTADTSTSTAYMQLSSLRAEDIAVYFCASGRPDYWGQGTTLTVSS

SEQ ID NO: 100 - protein, 114 aa, Artificial Sequence
  RA4 Caninised Heavy Chain - VHC6
QVQLVQSGAEVKKPGESVKVSCKTSGYTFTDYSMHWVRQAPGKGLDWMGWINTETGKTIHAEDFKGRVTLTADTSTSTAYMELSSLRAGDIAVYFCASGRPDYWGQGTTLTVSS

SEQ ID NO: 101 - protein, 111 aa, Artificial Sequence
  RA4 Caninised Light Chain - VLC6
EIVLTQSPASLSLSQGEKVTITCRASESVEYYGTSLMQWYQQKPGQAPKLLIYAASGVESGVPSRFSGSGSGTDFSFTISSLEPEDVAVYFCQQSRKVPYTFGGGTKLEIK

(19) United States
(12) Patent Application Publication
(10) Pub. No.: US 2023/0416356 A1
(43) Pub. Date: Dec. 28, 2023

(54) ANTIGEN-BINDING MOLECULES AND USES THEREOF

(71) Applicant: Scout Bio, Inc., Philadelphia, PA (US)

(72) Inventors: Samantha J. BUSFIELD, Philadelphia, PA (US); Matthew J. WILSON, Philadelphia, PA (US)

(21) Appl. No.: 18/037,938

(22) PCT Filed: Nov. 22, 2021
(86) PCT No.: PCT/US2021/060369

§ 371 Date: May 19, 2023

Related U.S. Application Data
(60) Provisional application No. 63/117,383, filed on Nov. 23, 2020.

(51) Int. Cl. C07K 16/24; C12N 15/86; A61P 37/02

(52) U.S. Cl. CPC C07K 16/244; C12N 15/86; A61P 37/02; C07K 2317/565; C07K 2317/20; C07K 2317/567; C12N 2750/14143

(57) ABSTRACT

The present disclosure relates to an antigen-binding molecule that specifically binds to interleukin-31 (IL-31), wherein the antigen-binding molecule comprises an immunoglobulin heavy chain variable domain and an immunoglobulin light chain variable domain.

### RELATED APPLICATIONS

This application claims priority to, and the benefit of, U.S. Provisional Application No. 63/117,383, filed Nov. 23, 2020. The contents of the aforementioned patent application are incorporated herein by reference in its entirety.

### FIELD OF INVENTION

The invention relates generally to antigen-binding molecules. In particular, the invention relates to antigen-binding molecules that specifically bind to and neutralise the activity of interleukin 31 (IL-31) and uses thereof for the treatment of conditions associated with abnormal IL-31 expression and/or activity, such as chronic inflammation, atopic dermatitis, eczema, pruritis, airway hypersensitivity and inflammatory bowel syndrome.

### SEQUENCE LISTING

The instant application contains a Sequence Listing which has been submitted in ASCII format via EFS-Web and is hereby incorporated by reference in its entirety. Said ASCII copy, created on Nov. 22, 2021, is named “SCTB-012-01WO_SeqList_ST25.txt” and is 61 KB in size.

### BACKGROUND

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

As discussed by Sinem Ba\check{g}ci and Ruzicka (2018, J. Allergy Clin. Immunol.; 141(3):858-866), IL-31 is a cytokine that is expressed in many human tissues and has been implicated in Th2-biased inflammatory responses. IL-31 signals through a receptor complex consisting of IL-31 receptor α and oncostatin M receptor β. Studies have shown that IL-31 is strongly linked with chronic pruritic skin disorders, such as atopic eczema, and represents a novel target for directed drug therapy.

IL-31 belongs to the glycoprotein 130 (gp130)/IL-6 cytokine family, which also include leukemia inhibitory factor, oncostatin M (OSM), cardiotrophin-1, ciliary neurotrophic factor, cardiotrophin-like cytokine, IL-6, and IL-11. Members of this family share the common chain of gp130 in their multiunit receptor and are involved in neuronal growth, bone metabolism, cardiac development, and immune regulation, such as T-cell differentiation. In humans, the gene encoding IL-31 is located on chromosome 12q24.31, and is composed of an open reading frame encoding a 164-amino-acid precursor and a predicted 141-amino-acid mature polypeptide containing the 4 a-helix structure. IL-31 has also been implicated in non-dermatological conditions, such as allergic asthma and rhinitis, inflammatory bowel diseases, malignancies (e.g., cancer), and osteoporosis.

From a therapeutic perspective, one of the first human trials investigating the blockade of IL-31 signaling was undertaken in healthy subjects and patients with atopic dermatitis with the humanized anti-human IL-31RA monoclonal antibody, nemolizumab (CIM331). The study showed that a single subcutaneous dose of nemolizumab significantly reduced visual analogue scale (VAS) scores for pruritus and the use of topical steroids, as well as an increase in sleep efficiency, although there were reports of adverse events, including exacerbation of atopic dermatitis and peripheral edema, in particular in patients receiving nemolizumab as compared to those receiving placebo.

Thus, there remains an urgent need for improved therapeutic strategies for treating conditions associated with excess IL-31 levels and/or activity, such as pruritis and other dermatological and non-dermatological conditions.

### SUMMARY

The present disclosure is predicated, at least in part, on an anti-IL-31 binding molecule comprising complementarity determining regions (CDR) that are capable of binding specifically to native IL-31 and whose framework regions can be modified for compatibility with a target species without loss of binding specificity and selectivity to native IL-31. The IL-31-binding molecules disclosed herein are therefore amenable to use in the treatment and prevention of conditions associated with abnormal IL-31 levels and/or activity, illustrative examples of which include pruritis and atopic dermatitis.

Thus, in an aspect disclosed herein, there is provided an antigen-binding molecule that specifically binds to interleukin-31 (IL-31), wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO:6 or an amino acid sequence having at least 80% sequence identity thereto, a VH CDR2 comprising the amino acid sequence of SEQ ID NO:7 or an amino acid sequence having at least 80% sequence identity thereto and a VH CDR3 comprising the amino acid sequence of SEQ ID NO:8 or an amino acid sequence having at least 80% sequence identity thereto; and wherein the VL comprises a complementarity determining region 1 (VL CDR1) comprising the amino acid sequence of SEQ ID NO:9 or an amino acid sequence having at least 80% sequence identity thereto, a VL CDR2 comprising the amino acid sequence of SEQ ID NO:10 or an amino acid sequence having at least 80% sequence identity thereto, and a VL CDR3 comprising the amino acid sequence of SEQ ID NO:11 or an amino acid sequence having at least 80% sequence identity thereto, wherein the amino acid sequences of the CDR are based on IMGT numbering.

The present disclosure is also predicated, at least in part, on the inventor's finding that the IL-31 binding molecules disclosed herein bind specifically to a region of canine IL-31 that has not been identified as the binding region of known anti-IL-31 antibody molecules. Thus, in another aspect disclosed herein, there is provided an antigen-binding molecule that does not compete for binding to IL-31 with (i) an IL-31 binding molecule that specifically binds to a region of IL-31 that corresponds to amino acid positions 13, 15, 20 and 26 of SEQ ID NO:1 or (ii) an IL-31 binding molecule that specifically binds to a region of IL-31 that corresponds to amino acid positions 76, 77, 81, and 84 of SEQ ID NO:1.

The present disclosure also extends to nucleic acid sequences encoding the antigen-binding molecules described herein, including their heavy and light chain sequences and IL-31-binding fragments thereof.

Thus, in one aspect, there is provided an isolated nucleic acid molecule comprising a nucleic acid sequence encoding the antigen-binding molecule described herein.

Also disclosed herein is an expression construct comprising a nucleic acid sequence encoding the antigen-binding molecule described herein. The nucleic acid sequence may be operably linked to one or more regulatory sequences.

The present disclosure also extends to a host cell comprising the expression construct described herein.

The present disclosure also extends to vector comprising a nucleic acid sequence encoding the antigen-binding molecule described herein. In an embodiment, the vector is an AAV vector.

The present disclosure also extends to a pharmaceutical composition comprising the antigen-binding molecule described herein, and a pharmaceutically acceptable carrier.

In another aspect disclosed herein, there is provided a method of treating or preventing a condition associated with increased expression and/or increased activity of IL-31, the method comprising administering to a subject in need thereof the antigen-binding molecule, the vector or the pharmaceutical composition as described herein.

The present disclosure also extends to a kit comprising the antigen-binding molecule, the vector, or the pharmaceutical composition described herein.

In another aspect disclosed herein, there is provided use of the antigen-binding molecule or the vector described herein in the manufacture of a medicament for treating or preventing a condition associated with increased expression and/or increased activity of IL-31 in a subject in need thereof.

The present disclosure also extends to the antigen-binding molecule, the vector, or the pharmaceutical composition as described herein for use in the treatment or prevention of a condition associated with increased expression and/or increased activity of IL-31 in a subject in need thereof.

The present disclosure also extends to the antigen-binding molecule, the vector, or the pharmaceutical composition as described herein for use in the treatment or prevention of a tumour induced to proliferate by IL-31 and conditions associated therewith in a subject in need thereof.

The present disclosure also extends to an antigen-binding molecule that specifically binds to an epitope of canine IL-31 comprising amino acid residues L29, Y32, Q33 and P40 of SEQ ID NO:1.

The present disclosure also extends to an antigen-binding molecule that competes for binding to canine IL-31 of SEQ ID NO:1 with the antigen-binding molecule described herein.

The present disclosure also extends to an immunogenic composition capable of raising an immune response to IL-31, wherein the composition comprises (i) an immunogen comprising a B cell epitope of IL-31 and (ii) a pharmaceutically acceptable carrier, wherein the B cell epitope comprises an amino acid sequence corresponding to amino acid positions 29-40 of SEQ ID NO:1, or an amino acid sequence that has at least 80% sequence identity thereto, and wherein the immunogen does not comprise the amino acid sequence of a native IL-31 molecule.

### BRIEF DESCRIPTION OF DRAWINGS

Embodiments of the present invention are hereafter described, by way of non-limiting example only, with reference to the accompanying drawings in which:

FIG. 1 shows binding of RA4 to canine IL-31. A dose response curve showing binding of the chimeric monoclonal antibody RA4 (chRA4) to canine IL-31 as determined by ELISA. Results shown are the ratio of absorbance at 405 nm of specific binding over background.

FIG. 2 shows inhibition of canine IL-31-induced STAT-3 phosphorylation in canine DH82 cells. Different concentrations of the chRA4 monoclonal antibody were pre-incubated with canine IL-31 prior to addition to DH82 cells. These data shows that RA4 can potently inhibit IL-31-induced STAT-3 phosphorylation in a dose-dependent manner. The observed IC₅₀ was 1.5 μg/mL.

FIG. 3 shows binding of anti-IL-31 antibodies chRA4, M14 and 34D03 to canine IL-31 (calL-31), feline IL-31 (felL-31), canine IL-31 Mutants 1 and canine IL-31 Mutant 2, as determined by ELISA. The data show that the chimeric RA4 binds equally well to canine IL-31, feline IL-31 and to the two canine IL-31 mutants (Mutants 1 and 2). M14 binds to canine IL-31 and to canine IL-31 Mutant 1, but not to canine IL-31 Mutant 2. M14 binding to feline IL-31 was also diminished. 34D03 also binds to canine IL-31 and to a lesser extent, feline IL-31, but did not bind to either of the canine IL-31 mutant proteins.

FIG. 4A shows a schematic representation of the canine/human chimeric proteins ca/huIL-31_c123_h4, cahuIL-31_c12_h34 and cahuIL-31_c1_h234. FIG. 4B shows the dose response curve of monoclonal antibody RA4 binding to canine IL-31 (calL-31), human IL-31 (hIL-31) and the chimeric canine/human IL-31 proteins (c123_h4, c12_h34 and c1_h234), as determined by ELISA.

FIG. 5A shows a schematic representation of truncated series of overlapping peptides covering the N-terminal sequence of canine IL-31. FIG. 5B shows the binding of RA4 and two previously described anti-IL-31 monoclonal antibodies, M14 and 34D03, to full length canine IL-31 (Full ca IL-31) and the overlapping peptides derived from the N-terminal of canine IL-31, as determined by ELISA.

FIG. 6 shows the identification of residues in the N-terminal region of canine IL-31 that are important for RA4 mAb binding. An alanine scanning library of a 25 amino acid region of the N-terminal domain of canine IL-31 was generated. Binding of RA4 mAb to each of the modified (alanine substituted) peptides was assessed by ELISA. Binding to each of the modified peptides was normalised to binding to the first peptide in the series to the far left of the X-axis (isoleucine).

FIG. 7 shows that RA4 mAb binds to a distinct and unique region of the N-terminal of canine IL-31. Full-length canine IL-31 proteins with either a single alanine substitution or a combination of all four alanine substitutions were used to evaluate the binding of RA4 mAb (A) or M14 mAb (B) by ELISA. Wild-type canine IL-31 (calL-31; SEQ ID NO:1) was included as a control.

### DETAILED DESCRIPTION

As described elsewhere herein, the present disclosure is predicated, at least in part, on an anti-IL-31 binding molecule comprising complementarity determining regions (CDR) that are capable of binding specifically to native IL-31 and whose framework regions can be modified for compatibility with a target species without loss of binding specificity and selectivity to native IL-31. The IL-31-binding molecules disclosed herein are therefore amenable to use in the treatment and prevention of conditions associated with abnormal IL-31 levels and/or activity, illustrative examples of which include pruritis and atopic dermatitis.

Thus, disclosed herein is an antigen-binding molecule that specifically binds to interleukin-31 (IL-31), wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO:6 or an amino acid sequence having at least 80% sequence identity thereto, a VH CDR2 comprising the amino acid sequence of SEQ ID NO:7 or an amino acid sequence having at least 80% sequence identity thereto and a VH CDR3 comprising the amino acid sequence of SEQ ID NO:8 or an amino acid sequence having at least 80% sequence identity thereto; and wherein the VL comprises a complementarity determining region 1 (VL CDR1) comprising the amino acid sequence of SEQ ID NO:9 or an amino acid sequence having at least 80% sequence identity thereto, a VL CDR2 comprising the amino acid sequence of SEQ ID NO:10 or an amino acid sequence having at least 80% sequence identity thereto, and a VL CDR3 comprising the amino acid sequence of SEQ ID NO:11 or an amino acid sequence having at least 80% sequence identity thereto, wherein the amino acid sequences of the CDR are based on IMGT numbering.

The CDR sequences disclosed herein are based on IMGT numbering, unless stated otherwise.

The present disclosure is also predicated, at least in part, on the inventors' finding that the IL-31 binding molecules disclosed herein bind specifically to a region of IL-31 that has not been identified for known IL-31 binding molecules. Thus, in an aspect disclosed herein, there is provided an antigen-binding molecule that does not compete for binding to IL-31 with (i) an IL-31 binding molecule that specifically binds to a region of IL-31 that corresponds to amino acid positions 13, 15, 20 and 26 of SEQ ID NO:1 or (ii) an IL-31 binding molecule that specifically binds to a region of IL-31 that corresponds to amino acid positions 76, 77, 80, 81, and 84 of SEQ ID NO:1.

The term “corresponds to” refers to the region of an IL-31 peptide sequence that, when aligned with the canine IL-31 peptide sequence of SEQ ID NO:1, comprises amino acid residues that align with the residues of SEQ ID NO:1. For example, a region of IL-31 that corresponds to amino acid positions 13, 15, 20 and 26 of SEQ ID NO:1 refers to the region of an IL-31 peptide sequence that, when aligned with the canine IL-31 peptide sequence of SEQ ID NO:1, comprises amino acid residues that align with the residues at positions 13, 15, 20 and 26 of SEQ ID NO:1. Similarly, a region of IL-31 that corresponds to amino acid positions 76, 77, 80, 81, and 84 of SEQ ID NO:1 refers to the region of an IL-31 peptide sequence that, when aligned with the canine IL-31 peptide sequence of SEQ ID NO:1, comprises amino acid residues that align with the residues at positions 76, 77, 80, 81, and 84 of SEQ ID NO:1.

As described elsewhere herein, the present inventors have also found that the IL-31 binding molecules disclosed herein bind to an epitope of IL-31 comprising amino acid residues L29, Y32, Q33 and P40 of SEQ ID NO:1. Thus, disclosed herein is an antigen-binding molecule that specifically binds to an epitope of canine IL-31 comprising amino acid residues L29, Y32, Q33 and P40 of SEQ ID NO:1.

The present disclosure also extends to an antigen-binding molecule that competes for binding to canine IL-31 of SEQ ID NO:1 with the antigen-binding molecule described herein.

The term “competes with”, as used herein, denotes that the two or more antigen-binding molecules compete for binding to an antigen (e.g., IL-31). In one exemplary assay, IL-31 is coated on a solid substrate and allowed to bind a first antigen-binding molecule, after which a second antigen-binding molecule is added. If the presence of the first antigen-binding molecule reduces binding of the second antigen-binding molecule to the IL-31, then the antigen-binding molecule is identified as competing for binding to the IL-31. The term “competes with” also includes combinations of antigen-binding molecules where one antigen-binding molecule reduces binding of another antigen-binding molecule, but where no competition is observed when the antigen-binding molecules are added in the reverse order. However, in some embodiments, the first and second antigen-binding molecules inhibit binding of each other to the antigen, regardless of the order in which they are added. In an embodiment, one antigen-binding molecule reduces binding of another antigen-binding molecule to the antigen by at least 20%, preferably by at least 30%, preferably by at least 40%, preferably by at least 50%, preferably by at least 60%, preferably by at least 70%, preferably by at least 80%, preferably by at least 90%, or more preferably by at least 95%.

The term “antigen-binding molecule”, as used herein, refers to a molecule that has binding specificity for a target antigen. It will be understood that this term extends to immunoglobulins, immunoglobulin fragments and non-immunoglobulin-derived protein frameworks that exhibit antigen-binding activity. Illustrative examples of suitable antigen-binding molecules include antibodies and antigen-binding fragments thereof. Preferably, the antigen-binding molecule binds specifically to IL-31 so as to neutralise, or substantially neutralise, its activity. The term “neutralise” is understood to mean that the antigen-binding molecule will bind to IL-31 and inhibit, reduce, abrogate, block or otherwise prevent the ability of the IL-31 molecule to bind to its native receptor. In some embodiments, the antigen-binding molecule will completely neutralise the activity of IL-31 (in vivo or in vitro) such that there is no or negligible IL-31 activity when compared to the absence of the antigen-binding molecule. In other embodiments, the antigen-binding molecule will partially neutralise the activity of IL-31 (in vivo or in vitro) such that there is less IL-31 activity when compared to the absence of the antigen-binding molecule.

The term “antibody”, as used herein, is understood to mean any antigen-binding molecule or molecular complex comprising at least one complementarity determining region (CDR) that binds specifically to, or interacts specifically with, the target antigen. The term “antibody” includes full-length immunoglobulin molecules comprising two heavy (H) chains and two light (L) chains inter-connected by disulfide bonds, as well as multimers thereof (e.g., IgM). Each heavy chain comprises a heavy chain variable region (which may be abbreviated as HCVR, VH or V_{H}) and a heavy chain constant region. The heavy chain constant region typically comprises three domains—C_{H}1, C_{H}2 and C_{H}3. Each light chain comprises a light chain variable region (which may be abbreviated as LCVR, VL, VK, V_{K} or V_{L}) and a light chain constant region. The light chain constant region will typically comprise one domain (C_{L}1). The V_{H} and V_{L} regions can be further subdivided into regions of hypervariability, termed complementarity determining regions (CDRs), interspersed with regions that are more conserved, also referred to as framework regions (FR). Each VH and VL typically comprises three CDRs and four FRs, arranged from amino-terminus to carboxy-terminus in the following order: FR1, CDR1, FR2, CDR2, FR3, CDR3, FR4. In some embodiments, the FRs of the antigen-binding molecules described herein may be identical to the FR of germline sequences of the target species (i.e., the species to which the antigen-binding molecules or antigen-binding fragments thereof, as described herein, will be administered). In some embodiments, the FR may be naturally or artificially modified. Whilst it is generally desirable that each of the FR sequences are identical to FR sequences derived from immunoglobulin molecules of the target species, including to minimize an immune response being raised against the binding molecule upon administration to a subject of the target species, in some embodiments, the antigen-binding molecule, or antigen-binding fragment thereof, may comprise one or more amino acid residues across one or more of its FR sequences that would be foreign at a corresponding position in one or more FR from the target species. Preferably, where the antigen-binding molecule, or antigen-binding fragment thereof, comprises one or more amino acid residues across one or more of its FR sequences that would be foreign at a corresponding position in the target species, that “foreign” amino acid residue will not (i) adversely impact the binding specificity of the antigen-binding molecule or antigen-binding fragment thereof to IL-31, including native IL-31 and/or (ii) cause an immune response to be raised against the antigen-binding molecule or to the antigen-binding fragment thereof when administered to a subject of the target species.

Suitable antibodies include canine IgGA, IgGB, IgGC and IgGD and feline IgG1 a, IgG1b and IgG2 (including sub-classes thereof). The subunit structures and three-dimensional configurations of different classes of immunoglobulins will be well known to persons skilled in the art.

As used herein, the term “complementarity determining region” (CDR) refers to the region of an immunoglobulin variable domain that recognizes and binds to the target antigen. Each variable domain may comprises up to three CDR sequences, identified as CDR1, CDR2 and CDR3. The amino acid sequence of each CDR is often defined by Kabat numbering (e.g., about residues 24-34 (L1), 50-56 (L2) and 89-97 (L3) of the light chain variable domain and residues 31-35 (H1), 50-65 (H2) and 95-102 (H3) of the heavy chain variable domain; Kabat et al., Sequences of Proteins of Immunological Interest, 5th Ed. Public Health Service, National Institutes of Health, Bethesda, Md. (1991)) and/or by Chothia numbering (e.g., about residues 26-32 (L1), 50-52 (L2) and 91-96 (L3) of the light chain variable domain and 26-32 (H1), 53-55 (H2) and 96-101 (H3) of the heavy chain variable domain; see Chothia and Lesk J. Mol. Biol. 196:901-917 (1987)). The CDR sequences disclosed herein are based on IMGT numbering, unless stated otherwise.

In an embodiment, the antigen-binding molecule, as described herein, is conjugated to another molecule or moiety, including functional moieties (e.g., toxins), detectable moieties (e.g., fluorescent molecules, radioisotopes), small molecule drugs and polypeptides.

It is to be understood that modifications may be made to the antigen-binding molecules described herein, including to the CDR sequences, without adversely impacting the binding specificity of the antigen-binding molecules described herein. Thus, the present disclosure extends to functional variants of the IL-31-binding molecules disclosed herein. The term “functional variant”, as used herein, is to be understood as meaning an IL-31-binding molecule comprising an amino acid sequence that differs from a parent or comparator sequence (.g., VH and/or VL) by one or more (e.g., by 1, 2, 3, 4, 5, 6, 7, 8, 9, 10, 11, 12, 14, 15, 16, 17, 18, 19, 20, 21 and so on) amino acid deletions, insertions and/or substitutions, wherein said difference does not, or does not completely, abolish the ability of the variant to bind to IL-31.

Suitable methods of determining whether a variant retains said function will be familiar to persons skilled in the art, illustrative examples of which are described elsewhere herein. A functional variant may comprise an amino acid sequence that differs from the comparator sequence (e.g., SEQ ID NOs:12 and 13). In some embodiments, the functional variant may comprise amino acid substitutions that enhance the binding affinity of the antigen-binding molecule to IL-31, as compared to the reference molecule to which the variation is compared (e.g., an antigen-binding molecule comprising the VH and VL or SEQ ID NOs:12 and 13). In an embodiment, the functional variant differs from the comparator by one or more conservative amino acid substitutions, as described elsewhere herein.

Reference to “at least 80% sequence identity” includes 80%, 85%, 86%, 87%, 88%, 89%, 90%, 91%, 92%, 93%, 94%, 95%, 96%, 97%, 98%, 99% or 100% sequence identity the reference sequence (e.g., of any one of SEQ ID NOs:6-13), for example, after optimal alignment or best fit analysis. Thus, in an embodiment, the functional variant of the antigen-binding molecule comprises, consists or consists essentially of an amino acid sequence that has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to SEQ ID NO:12 or SEQ ID NO:13, for example, after optimal alignment or best fit analysis. In another embodiment, the CDR sequences of the functional variant of the antigen-binding molecule comprise, consist or consist essentially of amino acid sequences having at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to any one of SEQ ID NOs:6-11, for example, after optimal alignment or best fit analysis.

In an embodiment, (i) the VH comprises a complementarity determining region 1 (VH CDR1) comprising an amino acid sequence that has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to SEQ ID NO:6, a VH CDR2 comprising an amino acid sequence that has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to SEQ ID NO:7 and a VH CDR3 comprising an amino acid sequence that has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to SEQ ID NO:8; and (ii) the VL comprises a complementarity determining region 1 (VL CDR1) comprising an amino acid sequence that has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to SEQ ID NO:9, a VL CDR2 comprising an amino acid sequence that has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to SEQ ID NO:10, and a VL CDR3 comprising an amino acid sequence that has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to SEQ ID NO:11.

In an embodiment, the VH comprises a complementarity determining region 1 (VH CDR1) comprising an amino acid sequence that has at least 80% sequence identity to SEQ ID NO:6, a VH CDR2 comprising an amino acid sequence that has at least 80% sequence identity to SEQ ID NO:7 and a VH CDR3 comprising an amino acid sequence that has at least 80% sequence identity to SEQ ID NO:8; and the VL comprises a complementarity determining region 1 (VL CDR1) comprising an amino acid sequence that has at least 80% sequence identity to SEQ ID NO:9, a VL CDR2 comprising an amino acid sequence that has at least 80% sequence identity to SEQ ID NO:10, and a VL CDR3 comprising an amino acid sequence that has at least 80% sequence identity to SEQ ID NO:11.

In an embodiment, the VH comprises a complementarity determining region 1 (VH CDR1) comprising, consisting or consisting essentially of an amino acid sequence of SEQ ID NO:6, a VH CDR2 comprising, consisting or consisting essentially of an amino acid of SEQ ID NO:7 and a VH CDR3 comprising, consisting or consisting essentially of an amino acid sequence of SEQ ID NO:8; and the VL comprising, consisting or consisting essentially of a complementarity determining region 1 (VL CDR1) comprising, consisting or consisting essentially of an amino acid sequence of SEQ ID NO:9, a VL CDR2 comprising, consisting or consisting essentially of an amino acid sequence of SEQ ID NO:10, and a VL CDR3 comprising, consisting or consisting essentially of an amino acid sequence of SEQ ID NO:11.

In an embodiment, the antigen-binding molecule described herein comprises a heavy chain variable region (VH) comprising the amino acid sequence of SEQ ID NO:12 or an amino acid sequence having at least 80%, preferably at least 85%, preferably at least 90%, preferably at least 95%, or more preferably at least 98% sequence identity thereto. In an embodiment, the antigen-binding molecule described herein comprises a light chain variable region (VH) comprising the amino acid sequence of SEQ ID NO:13 or an amino acid sequence having at least 80%, preferably at least 85%, preferably at least 90%, preferably at least 95%, or more preferably at least 98% sequence identity thereto.

In an embodiment, the antigen-binding molecule described herein comprises (a) a heavy chain variable region (VH) comprising the amino acid sequence of SEQ ID NO:12 or an amino acid sequence having at least 80%, preferably at least 85%, preferably at least 90%, preferably at least 95%, or more preferably at least 98% sequence identity thereto, and (b) a light chain variable region (VL) comprising the amino acid sequence of SEQ ID NO:13 or an amino acid sequence having at least 80%, preferably at least 85%, preferably at least 90%, preferably at least 95%, or more preferably at least 98% sequence identity thereto.

In an embodiment, the antigen-binding molecule described herein comprises (a) a heavy chain variable region (VH) comprising the amino acid sequence of SEQ ID NO:12 or an amino acid sequence having at least 80% sequence identity thereto, and (b) a light chain variable region (VL) comprising the amino acid sequence of SEQ ID NO:13 or an amino acid sequence having at least 80% sequence identity thereto.

In an embodiment, the heavy chain variable region (VH) comprises the amino acid sequence of SEQ ID NO:12, and the light chain variable region (VL) comprises the amino acid sequence of SEQ ID NO:13.

In an embodiment, the heavy chain variable region (VH) consists or consists essentially of the amino acid sequence of SEQ ID NO:12, and the light chain variable region (VL) consists or consists essentially of the amino acid sequence of SEQ ID NO:13.

The terms “identity”, “similarity”, “sequence identity”, “sequence similarity”, “homology”, “sequence homology” and the like, as used herein, mean that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. This may be referred to as conservative substitution. In an embodiment, the amino acid sequences may be modified by way of conservative substitution of any of the amino acid residues contained therein, such that the modification has no effect on the binding specificity or functional activity of the modified polypeptide when compared to the unmodified polypeptide.

In some embodiments, sequence identity with respect to a peptide sequence relates to the percentage of amino acid residues in the candidate sequence which are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to persons skilled in the art. For example, the percentage of identity or similarity of two amino acid sequences can be readily calculated using algorithms, for example, BLAST, FASTA, or the Smith-Waterman algorithm.

Techniques for determining an amino acid sequence “similarity” are well known to persons skilled in the art. In general, “similarity” means an exact amino acid to amino acid comparison of two or more peptide sequences or at the appropriate place, where amino acids are identical or possess similar chemical and/or physical properties such as charge or hydrophobicity. A so-termed “percent similarity” then can be determined between the compared peptide sequences. In general, “identity” refers to an exact amino acid to amino acid correspondence of two peptide sequences.

Two or more peptide sequences can also be compared by determining their “percent identity”. The percent identity of two sequences may be described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). Suitable programs for calculating the percent identity or similarity between sequences are generally known in the art.

Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

In some embodiments, the functional variant comprises at least one (e.g., 1, 2, 3, 4 or 5) amino acid substitutions, preferably conservative amino acid substitutions, in any one or more of the CDR sequences. In an embodiment, the antigen-binding molecule comprises 1, 2 or 3 amino acid substitutions in any one or more of the CDR sequences. In other embodiments, the functional variant comprises one or more amino acid substitutions, insertions and/or deletions in the non-CDR regions (e.g., in the framework regions) of the VH and/or VL sequences.

It will be well within the ability of persons skilled in the art to screen for and identity functional variants capable of specifically binding to IL-31. An illustrative example of a suitable screening method is described elsewhere herein.

In a preferred embodiment, the amino acid substitutions are conservative amino acid substitutions. A “conservative amino acid substitution” is to be understood as meaning a substitution in which the amino acid residue is replaced with an amino acid residue having a similar side chain. Families of amino acid residues having similar side chains have been defined in the art, which can be generally sub-classified as shown in the table “Amino Acid Classification”, below:

### Amino Acid Sub-Classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity.

Conservative substitutions are also shown in the table below (EXEMPLARY AND PREFERRED AMINO ACID SUBSTITUTIONS). Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants can be screened for their ability to bind specifically to IL-31 using methods known to persons skilled in the art, including those methods described elsewhere herein.

### Exemplary and Preferred Amino Acid Substitutions

| | | Preferred |
|---|---|---|
| Original Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

In an embodiment, the antigen-binding molecules described herein bind specifically to canine IL-31. In an embodiment, the antigen-binding molecules described herein bind specifically to feline IL-31.

The present disclosure extends to antigen binding molecules that bind specifically to native IL-31 (i.e., naturally-occurring IL-31), as well as to variants thereof. Such variants may include IL-31 molecules that differ from a naturally-occurring (wild-type) molecule by one or more amino acid substitutions, deletions and/or insertions. Variant IL-31 molecules of this type may be naturally-occurring or synthetic (e.g., recombinant) forms. It is to be understood, however, that in a preferred embodiment, the antigen-binding molecules described herein bind specifically to a native form of IL-31.

The terms “antigen-binding fragment”, “antigen-binding portion”, “antigen-binding domain”, “antigen-binding site” and the like are used interchangeably herein to refer to a part of an antigen-binding molecule that retains the ability to bind to the target antigen; that is, to IL-31, including native IL-31. These terms include naturally occurring, enzymatically obtainable, synthetic or genetically engineered (recombinant) polypeptides and glycoproteins that specifically bind to IL-31 to form a complex.

Antigen-binding fragments may be derived, for example, from naturally-derived immunoglobulin molecules using any suitable method known to persons skilled in the art, illustrative examples of which include proteolytic digestion or recombinant genetic engineering techniques involving the manipulation and expression of nucleic acid sequences encoding antibody variable and optionally constant domains. Suitable nucleic acid sequences are known and/or are readily available from, e.g., commercial sources, DNA libraries (including, e.g., phage-antibody libraries), or can be synthesized. The nucleic acid sequences may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of suitable antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g., monovalent nanobodies, bivalent nanobodies, etc.), and small modular immunopharmaceuticals (SMIPs), are also encompassed by the term “antigen-binding fragment,” as used herein.

In an embodiment, an antigen-binding fragment comprises at least one immunoglobulin variable domain. The variable domain may comprise an amino acid sequence of any suitable length or composition and will generally comprise at least one CDR which is adjacent to or in frame with one or more framework sequences. Where the antigen-binding fragment comprises a V_{H} domain and a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In some embodiments, an antigen-binding fragment may comprise at least one variable domain covalently linked to at least one constant domain. Non-limiting configurations of variable and constant domains that may be found within an antigen-binding fragment include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3, (vi) V_{H}-C_{H}2-C_{H}3; (vii) V_{H}-C_{L}; (viii) V_{L}-C_{H}1; (ix) V_{L}-C_{H}2, (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. In some embodiments, the antigen-binding fragment, as herein described, may comprise a homo-dimer or hetero-dimer (or other multimer) of any of the variable and constant domain configurations listed above in non-covalent association with one another and/or with one or more monomeric VH or VL domains (e.g., by disulfide bond(s)). A multispecific antigen-binding molecule will typically comprise at least two different variable domains, wherein each variable domain is capable of specifically binding to a separate antigen or to a different epitope on the same antigen. Any multispecific antigen-binding molecule format, including bispecific antigen-binding molecule formats, may be adapted for use in the context of an antigen-binding fragment of an antibody of the present disclosure using routine techniques available in the art.

The term “variable region” or “variable domain” refers to the domain of an immunoglobulin heavy or light chain that is involved in binding to the target antigen. The variable domains of the heavy chain and light chain (VII and VL, respectively) of a native immunoglobulin molecule will generally have similar structures, with each domain comprising four conserved framework regions and three hypervariable regions (HVRs). See, e.g., Kindt et al., Kuby Immunology, 6th ed., W.H. Freeman and Co., page 91 (2007). A single V_{H} or V_{L} domain may be sufficient to confer antigen-binding specificity.

The antigen-binding molecules described herein may suitably be modified for compatibility with the target species, for example, to minimise or otherwise avoid an immune response being generated towards the antigen-binding molecule following administration to that species. In an embodiment, the antigen-binding molecule is caninized, felinized or equinized.

By “caninized” is meant that the antigen-binding molecule comprises an amino acid sequence that is compatible with canine, such that the amino acid sequence is unlikely to be seen as foreign by the immune system of a canine subject. In an embodiment, the caninized antigen-binding molecule comprises one or more immunoglobulin framework regions derived from one or more canine immunoglobulin molecules. In some embodiments, all of the framework regions of the caninized antigen-binding molecule will be derived from one or more canine immunoglobulin molecules. The caninized antibody may optionally comprise an immunoglobulin heavy chain constant region derived from a canine immunoglobulin molecule. Thus, in an embodiment, the antigen-binding molecule is a caninized antigen-binding molecule.

By “felinized” is meant that the antigen-binding molecule comprises an amino acid sequence that is compatible with feline, such that the amino acid sequence is unlikely to be seen as foreign by the immune system of a feline subject. In an embodiment, the felinized antigen-binding molecule comprises one or more immunoglobulin framework regions derived from one or more feline immunoglobulin molecules. In some embodiments, all of the framework regions of the felinized antigen-binding molecule will be derived from one or more feline immunoglobulin molecules. The felinized antibody may optionally comprise an immunoglobulin heavy chain constant region derived from a feline immunoglobulin molecule. In an embodiment, the antigen-binding molecule is a felinized antigen-binding molecule.

By “equinized” is meant that the antigen-binding molecule comprises an amino acid sequence that is compatible with equine, such that the amino acid sequence is unlikely to be seen as foreign by the immune system of an equine subject. In an embodiment, the equinized antigen-binding molecule comprises one or more immunoglobulin framework regions derived from one or more equine immunoglobulin molecules. In some embodiments, all of the framework regions of the equinized antigen-binding molecule will be derived from one or more equine immunoglobulin molecules. The equinized antibody may optionally comprise an immunoglobulin heavy chain constant region derived from an equine immunoglobulin molecule.

It is to be understood that the present disclosure also extends to antigen-binding molecules that are compatible with species other than canine, feline and equine. In this context, the antigen-binding molecules can be referred to as “speciesized”, referring to the target species to which the molecule will be administered.

Suitable methods of designing and producing recombinant antibodies or antigen-binding molecules that are compatible with the target species will be familiar to persons skilled in the art, illustrative examples of which are described in Cattaneo (2010; Curr. Op. Mol. Ther. 12(1):94-106), WO 2006/131951, WO 2012/153122, WO 2013/034900, WO 2012/153121 and WO 2012/153123, the contents of which are incorporated herein by reference in their entirety.

In an embodiment, the antigen-binding molecule is caninized. In an embodiment, the caninized antigen-binding molecule comprises: (i) a heavy chain variable domain (VH) comprising:
(a) a heavy chain variable domain framework region 1 (VHFR1) amino acid sequence having at least 80% sequence identity to a VHFR1 amino acid sequence selected from the group consisting of SEQ ID NOs:18, 22, 26, 30 and 34,
(b) a VHFR2 amino acid sequence having at least 80% sequence identity to a VHFR2 amino acid sequence selected from the group consisting of SEQ ID NOs:19, 23, 27, 31 and 35,
(c) a VHFR3 amino acid sequence having at least 80% sequence identity to a VHFR3 amino acid sequence selected from the group consisting of SEQ ID NOs:20, 24, 27, 32 and 36, and
(d) a VHFR4 amino acid sequence having at least 80% sequence identity to a VHFR4 amino acid sequence selected from the group consisting of SEQ ID NOs:21, 25, 28, 33 and 37;
   and/or (ii) a light chain variable domain (VL) comprising:
(e) a heavy chain variable domain framework region 1 (VLFR1) amino acid sequence having at least 80% sequence identity to a VLFR1 amino acid sequence selected from the group consisting of SEQ ID NOs:38, 42, 46 and 50,
(f) a VLFR2 amino acid sequence having at least 80% sequence identity to a VLFR2 amino acid sequence selected from the group consisting of SEQ ID NOs:39, 43, 47 and 51,
(g) a VLFR3 amino acid sequence having at least 80% sequence identity to a VLFR3 amino acid sequence selected from the group consisting of SEQ ID NOs:40, 44, 48 and 52,
(h) a VLFR4 amino acid sequence having at least 80% sequence identity to a VHFR4 amino acid sequence selected from the group consisting of SEQ ID NOs:41, 45, 49 and 53.

In an embodiment, the caninized antigen-binding molecule comprises:
(i) a heavy chain variable domain (VH) comprising:
   (a) a heavy chain variable domain framework region 1 (VHFR1) amino acid sequence having at least 80% sequence identity to a VHFR1 amino acid sequence of SEQ ID NO: 81 or SEQ ID NO:85,
   (b) a VHFR2 amino acid sequence having at least 80% sequence identity to a VHFR2 amino acid sequence of SEQ ID NO: 82 or SEQ ID NO:86,
   (c) a VHFR3 amino acid sequence having at least 80% sequence identity to a VHFR3 amino acid sequence of SEQ ID NO: 83 or SEQ ID NO:87, and
   (d) a VHFR4 amino acid sequence having at least 80% sequence identity to a VHFR4 amino acid sequence of SEQ ID NO: 84 or SEQ ID NO:88; and/or
(ii) a light chain variable domain (VL) comprising:
   (e) a heavy chain variable domain framework region 1 (VLFR1) amino acid sequence having at least 80% sequence identity to a VLFR1 amino acid sequence of SEQ ID NO: 89,
   (f) a VLFR2 amino acid sequence having at least 80% sequence identity to a VLFR2 amino acid sequence of SEQ ID NO: 90,
   (g) a VLFR3 amino acid sequence having at least 80% sequence identity to a VLFR3 amino acid sequence of SEQ ID NO: 91, and
   (h) a VLFR4 amino acid sequence having at least 80% sequence identity to a VHFR4 amino acid sequence of SEQ ID NO: 92.

In an embodiment, the caninized antigen-binding molecule comprises a heavy chain variable domain (VH) comprising an amino acid sequence having at least 80% sequence identity to a VH amino acid sequence of SEQ ID NO: 99 or SEQ ID NO:100.

In an embodiment, the caninized antigen-binding molecule comprises a light chain variable domain (VL) comprising an amino acid sequence having at least 80% sequence identity to a VL amino acid sequence of SEQ ID NO: 101.

In an embodiment, the antigen-binding molecule is felinized. In an embodiment, the felinized antigen-binding molecule comprises: (i) a heavy chain variable domain (VH) comprising:
(a) a heavy chain variable domain framework region 1 (VHFR1) amino acid sequence having at least 80% sequence identity to a VHFR1 amino acid sequence of SEQ ID NO:54,
(b) a VHFR2 amino acid sequence having at least 80% sequence identity to a VHFR2 amino acid of SEQ ID NO:55,
(c) a VHFR3 amino acid sequence having at least 80% sequence identity to a VHFR3 amino acid sequence of SEQ ID NO:56, and
(d) a VHFR4 amino acid sequence having at least 80% sequence identity to a VHFR4 amino acid sequence of SEQ ID NO:57;
   and/or (ii) a light chain variable domain (VL) comprising:
(e) a light chain variable domain framework region 1 (VLFR1) amino acid sequence having at least 80% sequence identity to a VLFR1 amino acid sequence of SEQ ID NO:58,
(f) a VLFR2 amino acid sequence having at least 80% sequence identity to a VLFR2 amino acid sequence of SEQ ID NO:59,
(g) a VLFR3 amino acid sequence having at least 80% sequence identity to a VLFR3 amino acid sequence of SEQ ID NO:60, and
(h) a VLFR4 amino acid sequence having at least 80% sequence identity to a VHFR4 amino acid sequence of SEQ ID NO:61.

Other suitable framework region sequences that can be employed when modifying the antigen-binding molecules disclosed herein for compatibility with a target species, including canine, feline and equine, will be familiar to persons skilled in the art, illustrative examples of which are described in in Cattaneo (2010; supra), WO 2006/131951, WO 2012/153122, WO 2013/034900, WO 2012/153121 and WO 2012/153123.

The phrase “specifically binds” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule binds to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules (e.g., immunoglobulins)[such that they are specifically immunoreactive with a particular antigen. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W.H. Freeman and Co. 1976)).

“Affinity” or “binding affinity” refers to the strength of the sum total of non-covalent interactions between a single binding site of a molecule (e.g., an antigen-binding molecule) and its binding partner (e.g., an antigen). Unless indicated otherwise, as used herein, “binding affinity” refers to intrinsic binding affinity which reflects a 1:1 interaction between members of a binding pair e.g., an antigen-binding molecule. The affinity of a molecule X for its partner Y can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide.

As used herein, the term “modified antibody” includes synthetic forms of antibodies which are altered such that they are not naturally occurring, e.g., antibodies that comprise at least two heavy chain portions but not two complete heavy chains (such as domain deleted antibodies or minibodies); multispecific forms of antibodies (e.g., bispecific, trispecific, etc.) altered to bind to two or more different antigens or to different epitopes on a single antigen; heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen).

In an embodiment, antigen-binding molecule is an antibody or an antigen-binding fragment thereof, as described elsewhere herein. In an embodiment, the antigen-binding fragment is selected from the group consisting of a Fab fragment, scFab, Fab′, a single chain variable fragment (scFv) and a one-armed antibody.

In an embodiment, the antigen-binding molecule comprises a heavy chain and/or a light chain constant region of an immunoglobulin molecule. In an embodiment, the antigen-binding molecule comprises a heavy chain and/or a light chain constant region of a canine, feline or equine immunoglobulin molecule.

In an embodiment, the antigen-binding molecule comprises a heavy chain constant region of a canine immunoglobulin molecule. In an embodiment, the antigen-binding molecule comprises a light chain constant region of a canine immunoglobulin molecule. In yet an embodiment, the antigen-binding molecule comprises a heavy chain and a light chain constant region of a canine immunoglobulin molecule.

In an embodiment, the antigen-binding molecule comprises a heavy chain constant region of a canine immunoglobulin molecule selected from the group consisting of IgGA, IgGB, IgGC and IgGD. In an embodiment, the antigen-binding molecule comprises a light chain constant region of a canine immunoglobulin molecule selected from the group consisting of IgGA, IgGB, IgGC and IgGD. In an embodiment, the antigen-binding molecule comprises a heavy chain and a light chain constant region of a canine immunoglobulin molecule selected from the group consisting of IgGA, IgGB, IgGC and IgGD.

In an embodiment, the antigen-binding molecule comprises a heavy chain constant region of a canine IgGA molecule. In an embodiment, the antigen-binding molecule comprises a light chain constant region of a canine IgGA molecule. In an embodiment, the antigen-binding molecule comprises a heavy chain and a light chain constant region of a canine IgGA molecule.

In an embodiment, the antigen-binding molecule comprises a heavy chain constant region of a canine IgGB molecule. In an embodiment, the antigen-binding molecule comprises a light chain constant region of a canine IgGB molecule. In an embodiment, the antigen-binding molecule comprises a heavy chain and a light chain constant region of a canine IgGB molecule.

In an embodiment, the antigen-binding molecule comprises a heavy chain constant region of a canine IgGC molecule. In an embodiment, the antigen-binding molecule comprises a light chain constant region of a canine IgGC molecule. In an embodiment, the antigen-binding molecule comprises a heavy chain and a light chain constant region of a canine IgGC molecule.

In an embodiment, the antigen-binding molecule comprises a heavy chain constant region of a canine IgGD molecule. In an embodiment, the antigen-binding molecule comprises a light chain constant region of a canine IgGD molecule. In an embodiment, the antigen-binding molecule comprises a heavy chain and a light chain constant region of a canine IgGD molecule.

In an embodiment, the canine immunoglobulin molecule is a canine IgGA comprising, consisting or consisting essentially of the amino acid sequence of SEQ ID NO:2, or an amino acid sequence having at least 80% sequence identity thereto. In an embodiment, the canine IgGA comprises, consists or consists essentially of the amino acid sequence of SEQ ID NO:2.

In an embodiment, the antigen-binding molecule comprises an immunoglobulin heavy chain comprising, consisting or consisting essentially of the amino acid sequence of SEQ ID NO:16 or an amino acid sequence having at least 80% sequence identity thereto.

In an embodiment, the antigen-binding molecule comprises an immunoglobulin light chain comprising, consisting or consisting essentially of the amino acid sequence of SEQ ID NO:17 or an amino acid sequence having at least 80% sequence identity thereto.

In an embodiment, the antigen-binding molecule comprises an immunoglobulin heavy chain comprising, consisting or consisting essentially of the amino acid sequence of SEQ ID NO:16.

In an embodiment, the antigen-binding molecule comprises an immunoglobulin light chain comprising, consisting or consisting essentially of the amino acid sequence of SEQ ID NO:17.

In an embodiment, VH is encoded by the nucleic acid sequence of SEQ ID NO:14. In an embodiment, the VH is encoded by the nucleic acid sequence of SEQ ID NO:15.

In an embodiment, the chimeric antibody, or the IL-31-binding fragment thereof, comprises a heavy chain constant region of a feline immunoglobulin molecule selected from the group consisting of IgG1a, IgG1b and IgG2. In an embodiment, the chimeric antibody, or the IL-31-binding fragment thereof, comprises a light chain constant region of a feline immunoglobulin molecule selected from the group consisting of IgG1 a, IgG1b and IgG2. In an embodiment, the chimeric antibody, or the IL-31-binding fragment thereof, comprises a heavy chain and a light chain constant region of a feline immunoglobulin molecule selected from the group consisting of IgG1 a, IgG1b and IgG2.

In an embodiment, the chimeric antibody, or the IL-31-binding fragment thereof, comprises a heavy chain constant region of a feline IgG1a molecule. In an embodiment, the chimeric antibody, or the IL-31-binding fragment thereof, comprises a light chain constant region of a feline IgG1a molecule. In an embodiment, the chimeric antibody, or the IL-31-binding fragment thereof, comprises a heavy chain and a light chain constant region of a feline IgG1a molecule.

In an embodiment, the chimeric antibody, or the IL-31-binding fragment thereof, comprises a heavy chain constant region of a feline IgG1b molecule. In an embodiment, the chimeric antibody, or the IL-31-binding fragment thereof, comprises a light chain constant region of a feline IgG1b molecule. In an embodiment, the chimeric antibody, or the IL-31-binding fragment thereof, comprises a heavy chain and a light chain constant region of a feline IgG1b molecule.

In an embodiment, the chimeric antibody, or the IL-31-binding fragment thereof, comprises a heavy chain constant region of a feline IgG2 molecule. In an embodiment, the chimeric antibody, or the IL-31-binding fragment thereof, comprises a light chain constant region of a feline IgG2 molecule. In an embodiment, the chimeric antibody, or the IL-31-binding fragment thereof, comprises a heavy chain and a light chain constant region of a feline IgG2 molecule.

Also disclosed herein is a chimeric molecule comprising an IL-31-binding molecule, as herein described, and a heterologous moiety. In some embodiments, the heterologous moiety may be a detectable moiety, a half-life extending moiety, or a therapeutic moiety. Thus, as used herein, a “chimeric” molecule is one which comprises one or more unrelated types of components or contains two or more chemically distinct regions which can be conjugated to each other, fused, linked, translated, attached via a linker, chemically synthesized, expressed from a nucleic acid sequence, etc. For example, a peptide and a nucleic acid sequence, a peptide and a detectable label, unrelated peptide sequences, and the like. In embodiments in which the chimeric molecule comprises amino acid sequences of different origin, the chimeric molecule includes (1) polypeptide sequences that are not found together in nature (i.e., at least one of the amino acid sequences is heterologous with respect to at least one of its other amino acid sequences), or (2) amino acid sequences that are not naturally adjoined. For example, a “chimeric” antibody” as used herein refers to an antibody in which a portion of the heavy and/or light chain is derived from a particular source or species, while the remainder of the heavy and/or light chain is derived from a different source or species.

In an embodiment, the antigen-binding molecule described herein is an antibody or an IL-31-binding fragment thereof. In an embodiment, the antibody is a chimeric antibody. Illustrative examples of chimeric antibodies include antibody molecules in which the VH and VL sequences are derived from mouse and the heavy and light chain constant regions are derived from a species other than mouse (e.g., heavy and light chain constant regions derived from canine, feline, equine immunoglobulin molecules).

In an embodiment, the chimeric antibody, or the IL-31-binding fragment thereof, comprises a heavy chain constant region of a canine immunoglobulin molecule. In an embodiment, the chimeric antibody, or the IL-31-binding fragment thereof, comprises a light chain constant region of a canine immunoglobulin molecule. In yet an embodiment, the chimeric antibody, or the IL-31-binding fragment thereof, comprises a heavy chain and a light chain constant region of a canine immunoglobulin molecule.

In an embodiment, the chimeric antibody, or the IL-31-binding fragment thereof, comprises a heavy chain constant region of a canine immunoglobulin molecule selected from the group consisting of IgGA, IgGB, IgGC and IgGD. In an embodiment, the chimeric antibody, or the IL-31-binding fragment thereof, comprises a light chain constant region of a canine immunoglobulin molecule selected from the group consisting of IgGA, IgGB, IgGC and IgGD. In an embodiment, the chimeric antibody, or the IL-31-binding fragment thereof, comprises a heavy chain and a light chain constant region of a canine immunoglobulin molecule selected from the group consisting of IgGA, IgGB, IgGC and IgGD.

In an embodiment, the chimeric antibody, or the IL-31-binding fragment thereof, comprises a heavy chain constant region of a canine IgGA molecule. In an embodiment, the chimeric antibody, or the IL-31-binding fragment thereof, comprises a light chain constant region of a canine IgGA molecule. In an embodiment, the chimeric antibody, or the IL-31-binding fragment thereof, comprises a heavy chain and a light chain constant region of a canine IgGA molecule.

In an embodiment, the chimeric antibody, or the IL-31-binding fragment thereof, comprises a heavy chain constant region of a canine IgGB molecule. In an embodiment, the chimeric antibody, or the IL-31-binding fragment thereof, comprises a light chain constant region of a canine IgGB molecule. In an embodiment, the chimeric antibody, or the IL-31-binding fragment thereof, comprises a heavy chain and a light chain constant region of a canine IgGB molecule.

In an embodiment, the chimeric antibody, or the IL-31-binding fragment thereof, comprises a heavy chain constant region of a canine IgGC molecule. In an embodiment, the chimeric antibody, or the IL-31-binding fragment thereof, comprises a light chain constant region of a canine IgGC molecule. In an embodiment, the chimeric antibody, or the IL-31-binding fragment thereof, comprises a heavy chain and a light chain constant region of a canine IgGC molecule.

In an embodiment, the chimeric antibody, or the IL-31-binding fragment thereof, comprises a heavy chain constant region of a canine IgGD molecule. In an embodiment, the chimeric antibody, or the IL-31-binding fragment thereof, comprises a light chain constant region of a canine IgGD molecule. In an embodiment, the chimeric antibody, or the IL-31-binding fragment thereof, comprises a heavy chain and a light chain constant region of a canine IgGD molecule.

In an embodiment, the canine immunoglobulin molecule is a canine immunoglobulin gamma heavy chain A. In an embodiment, the canine immunoglobulin gamma heavy chain A comprises, consists or consists essentially of the amino acid sequence of SEQ ID NO:2, or an amino acid sequence having at least 80% sequence identity thereto. In an embodiment, the canine immunoglobulin gamma heavy chain A comprises, consists or consists essentially of the amino acid sequence of SEQ ID NO:2.

In an embodiment, the chimeric antibody comprises, consists or consists essentially of a heavy chain comprising the amino acid sequence of SEQ ID NO:16 or an amino acid sequence having at least 80% sequence identity thereto.

In an embodiment, the chimeric antibody comprises, consists or consists essentially of a light chain comprising the amino acid sequence of SEQ ID NO:17 or an amino acid sequence having at least 80% sequence identity thereto.

In an embodiment, the chimeric antibody comprises, consists or consists essentially of a heavy chain comprising the amino acid sequence of SEQ ID NO:16.

In an embodiment, the chimeric antibody comprises, consists or consists essentially of a light chain comprising the amino acid sequence of SEQ ID NO:17.

In an embodiment, VH is encoded by the nucleic acid sequence of SEQ ID NO:14. In an embodiment, the VH is encoded by the nucleic acid sequence of SEQ ID NO:15.

In another embodiment, the chimeric antibody, or the IL-31-binding fragment thereof, comprises a heavy chain constant region of a feline immunoglobulin molecule. In an embodiment, the chimeric antibody, or the IL-31-binding fragment thereof, comprises a light chain constant region of a feline immunoglobulin molecule. In yet an embodiment, the chimeric antibody, or the IL-31-binding fragment thereof, comprises a heavy chain and a light chain constant region of a feline immunoglobulin molecule.

In an embodiment, the chimeric antibody, or the IL-31-binding fragment thereof, comprises a heavy chain constant region of a feline immunoglobulin molecule selected from the group consisting of IgG1a, IgG1b and IgG2. In an embodiment, the chimeric antibody, or the IL-31-binding fragment thereof, comprises a light chain constant region of a feline immunoglobulin molecule selected from the group consisting of IgG1 a, IgG1b and IgG2. In an embodiment, the chimeric antibody, or the IL-31-binding fragment thereof, comprises a heavy chain and a light chain constant region of a feline immunoglobulin molecule selected from the group consisting of IgG1 a, IgG1b and IgG2.

In an embodiment, the chimeric antibody, or the IL-31-binding fragment thereof, comprises a heavy chain constant region of a feline IgG1a molecule. In an embodiment, the chimeric antibody, or the IL-31-binding fragment thereof, comprises a light chain constant region of a feline IgG1a molecule. In an embodiment, the chimeric antibody, or the IL-31-binding fragment thereof, comprises a heavy chain and a light chain constant region of a feline IgG1a molecule.

In an embodiment, the chimeric antibody, or the IL-31-binding fragment thereof, comprises a heavy chain constant region of a feline IgG1b molecule. In an embodiment, the chimeric antibody, or the IL-31-binding fragment thereof, comprises a light chain constant region of a feline IgG1b molecule. In an embodiment, the chimeric antibody, or the IL-31-binding fragment thereof, comprises a heavy chain and a light chain constant region of a feline IgG1b molecule.

In an embodiment, the chimeric antibody, or the IL-31-binding fragment thereof, comprises a heavy chain constant region of a feline IgG2 molecule. In an embodiment, the chimeric antibody, or the IL-31-binding fragment thereof, comprises a light chain constant region of a feline IgG2 molecule. In an embodiment, the chimeric antibody, or the IL-31-binding fragment thereof, comprises a heavy chain and a light chain constant region of a feline IgG2 molecule.

The present disclosure also extends to the use of heavy chain and light chain constant region sequences modified to improve, for example, stability and serum half-life.

Suitable heavy chain and light chain constant region sequences, including modified sequences of non-human species (e.g., canine, feline and equine) will be familiar to persons skilled in the art, illustrative examples of which are described in WO 2019/035010, the entire contents of which is incorporated herein by reference.

Also disclosed herein is an isolated nucleic acid molecule comprising a nucleic acid sequence encoding the antigen-binding molecule described herein. In an embodiment, there is provided an isolated nucleic acid molecule comprising (i) a nucleic acid sequence encoding an immunoglobulin heavy chain variable region having at least 80% sequence identity to SEQ ID NO:12 and/or (ii) a nucleic acid sequence encoding an immunoglobulin light chain variable region having at least 80% sequence identity to SEQ ID NO:13.

In an embodiment, the nucleic acid sequence encoding an immunoglobulin heavy chain variable region comprises the nucleic acid sequence of SEQ ID NO:14.

In another embodiment, the nucleic acid sequence encoding an immunoglobulin light chain variable region comprises the nucleic acid sequence of SEQ ID NO:15.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

Also disclosed herein is an expression vector that comprises a nucleic acid encoding the IL-31-binding molecules, as described herein.

By “vector” is meant a nucleic acid molecule, preferably a DNA molecule derived, for example, from a plasmid, bacteriophage, or virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

In an embodiment, the vector is an adeno-associated virus (AAV) vector that enables the IL-31-binding molecule, as described herein, to be safely administered to subjects and to provide a persistent expression of the IL-31-binding molecule in the subject.

Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61: 447-57 (1990)). These proteins also regulate transcription from endogenous AAV promoters and promoters within helper viruses (see, e.g., Pereira et al., J. Virol., 71: 1079-1088 (1997)). The other Rep proteins modify the function of Rep78 and Rep68. The cap genes encode the capsid proteins VP1, VP2, and VP3. The cap genes are transcribed from the p40 promoter.

Also disclosed herein is an expression construct comprising a nucleic acid sequence encoding the IL-31-binding molecule, as described herein, operably linked to one or more regulatory sequences.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements or regulatory sequences such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be contained within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

By “control element”, “control sequence”, “regulatory sequence” and the like, as used herein, is meant a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

Also disclosed herein is a host cell comprising the construct as defined herein.

The terms “host”, “host cell”, “host cell line” and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antigen binding molecules of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue. In one embodiment, the host cell is a CHO or HEK293 cell line.

Methods for producing a modified IL-31-binding molecule, as described herein, are also provided, such methods comprising culturing the host cell disclosed herein and recovering the IL-31-binding molecule from the host cell or culture medium.

Also disclosed herein is a pharmaceutical composition comprising the IL-31-binding molecule or a vector, as described herein, and a pharmaceutically acceptable carrier.

As noted elsewhere herein, the present inventors have found that the IL-31 binding molecules disclosed herein bind to an epitope of IL-31 that is not bound by any known IL-31 antigen-binding molecules; specifically, to an epitope of IL-31 comprising amino acid residues L29, Y32, Q33 and P40 of SEQ ID NO:1. Identification of this epitope of IL-31 advantageously allows the identified epitope, or a mimotope thereof, to be employed as an immunogenic composition for inducing an immune response when the immunogenic composition is administered to a subject. Thus, in an aspect disclosed herein, there is provided an immunogenic composition capable of raising an immune response to IL-31, wherein the composition comprises (i) an immunogen comprising a B cell epitope of IL-31 and (ii) a pharmaceutically acceptable carrier, wherein the B cell epitope comprises an amino acid sequence corresponding to amino acid positions 29-40 of SEQ ID NO:1, or an amino acid sequence that has at least 80% sequence identity thereto, and wherein the immunogen does not comprise the amino acid sequence of a native IL-31 molecule. In an embodiment, the immunogen consists of an amino acid sequence corresponding to amino acid positions 29-40 of SEQ ID NO:1, or an amino acid sequence that has at least 80% sequence identity thereto. In another embodiment, the immunogen consists of an amino acid sequence corresponding to amino acid positions 29-40 of SEQ ID NO:1. As noted elsewhere herein, reference to “at least 80% sequence identity” includes 80%, 85%, 86%, 87%, 88%, 89%, 90%, 91%, 92%, 93%, 94%, 95%, 96%, 97%, 98%, 99% or 100% sequence identity the reference sequence, for example, after optimal alignment or best fit analysis.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, colouring agents, wetting or emulsifying agents, pH buffering agents, preservatives, and the like.

Representative pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except, insofar as any conventional carrier is incompatible with the active ingredient(s), its use in the pharmaceutical compositions is contemplated.

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Suitable pharmaceutical compositions may be administered intravenously, subcutaneously or intramuscularly. In some embodiments, the compositions are in the form of injectable or infusible solutions. A preferred mode of administration is parenteral (e.g., intravenous, subcutaneous, intraperitoneal, intramuscular). In specific embodiments, the pharmaceutical composition is administered by intravenous infusion or injection. In other embodiments, the pharmaceutical composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin and/or by the maintenance of the required particle size. In specific embodiments, an agent of the present disclosure may be conjugated to a vehicle for cellular delivery. In these embodiments, the agent may be encapsulated in a suitable vehicle to either aid in the delivery of the agent to target cells, to increase the stability of the agent, or to minimize potential toxicity of the agent. As will be appreciated by a skilled artisan, a variety of vehicles are suitable for delivering an agent of the present disclosure. Non-limiting examples of suitable structured fluid delivery systems may include nanoparticles, liposomes, microemulsions, micelles, dendrimers and other phospholipid-containing systems. Methods of incorporating agents of the present disclosure into delivery vehicles are known in the art. Although various embodiments are presented below, it will be appreciate that other methods known in the art to incorporate an antigen-binding molecule, as described herein, into a delivery vehicle are contemplated.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. An antigen-binding molecule of the present disclosure can be administered on multiple occasions. Intervals between single dosages can be daily, weekly, monthly or yearly. Intervals can also be irregular as indicated by measuring blood levels of modified polypeptide or antigen in the patient. Alternatively, the antigen-binding molecule can be administered as a sustained release formulation, in which case less frequent administration is required. Dosage and frequency vary depending on the half-life of the polypeptide in the patient.

It may be advantageous to formulate compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutically acceptable carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Dosages and therapeutic regimens of the antigen-binding molecule can be determined by a skilled artisan. In certain embodiments, the antigen-binding molecule is administered by injection (e.g., subcutaneously or intravenously) at a dose of about 0.01 to 40 mg/kg, e.g., 0.01 to 0.1 mg/kg, e.g., about 0.1 to 1 mg/kg, about 1 to 5 mg/kg, about 5 to 25 mg/kg, about 10 to 40 mg/kg, or about 0.4 mg/kg. The dosing schedule can vary from e.g., once a week to once every 2, 3, or 4 weeks. In one embodiment, the antigen-binding molecule is administered at a dose from about 10 to 20 mg/kg every other week. An exemplary, non-limiting range for an effective amount of an antigen-binding molecule of the present disclosure is 0.01-5 mg/kg, more suitably 0.03-2 mg/kg.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include an effective amount of agent (i.e., the IL-31-binding molecule) disclosed herein. The effective amount may be a “therapeutically effective amount” or a “prophylactically effective amount”. A “therapeutically effective amount” refers to an amount effective, at dosages and for periods of time necessary, to achieve the desired therapeutic result. A therapeutically effective amount of the agent may vary according to factors such as the disease state, age, sex, and weight of the individual, and the ability of the agent to elicit a desired response in the individual. A therapeutically effective amount is also one in which any toxic or detrimental effects of the agent is outweighed by the therapeutically beneficial effects. Alternatively, this property of a composition can be evaluated by examining the ability of the compound to inhibit, for example in in vitro by assays known to the skilled practitioner.

By contrast, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also disclosed herein is a method of treating or preventing a condition associated with increased expression and/or increased activity of IL-31, the method comprising administering the antigen-binding molecule, the vector or the pharmaceutical composition, as described herein, to a subject in need thereof.

The term “treating” as used herein may refer to (1) delaying the appearance of one or more symptoms of the condition; (2) inhibiting the development of the condition or one or more symptoms of the condition; (3) relieving the condition, i.e., causing regression of the condition or at least one or more symptoms of the condition; and/or (4) causing a decrease in the severity of the condition or of one or more symptoms of the condition.

The terms “treating”, “treatment” and the like, are used interchangeably herein to mean relieving, reducing, alleviating, ameliorating or otherwise inhibiting the condition, including one or more symptoms of the condition. The terms “prevent”, “preventing”, “prophylaxis”, “prophylactic”, “preventative” and the like are used interchangeably herein to mean preventing or delaying the onset of the condition, or the risk of developing the condition.

The terms “treating”, “treatment” and the like also include relieving, reducing, alleviating, ameliorating or otherwise inhibiting the effects of the condition for at least a period of time. It is also to be understood that terms “treating”, “treatment” and the like do not imply that the condition, or a symptom thereof, is permanently relieved, reduced, alleviated, ameliorated or otherwise inhibited and therefore also encompasses the temporary relief, reduction, alleviation, amelioration or otherwise inhibition of the condition, or of a symptom thereof.

The terms “subject”, “patient”, “host” or “individual” used interchangeably herein, refer to any animal in need of treatment. The subject will typically encompass non-human subjects, including, but not limited to, mammals, birds and fish, and suitably encompasses domestic, farm, zoo and wild animals, such as, for example, cows, pigs, horses, goats, sheep or other hoofed animals, dogs, cats, chickens, ducks, non-human primates, guinea pigs, rabbits, ferrets, rats, hamsters and mice. In an embodiment, the subject is selected from the group consisting of a canine, a feline and an equine. In an embodiment, the subject is a canine. In an embodiment, the subject is a feline. In an embodiment, the subject is an equine.

Conditions associated with an abnormal (e.g., increased) level and/or abnormal (e.g., increased) activity of IL-31 will be familiar to persons skilled in the art, illustrative example of which include chronic pruritic skin disorders, such as atopic eczema, and non-dermatological conditions, such as allergic asthma and rhinitis, inflammatory bowel diseases, malignancies (e.g., cancer), and osteoporosis.

In an embodiment, the condition associated with increased expression and/or increased activity of IL-31 is selected from the group consisting of pruritis and dermatitis. In an embodiment, the condition is atopic dermatitis. In an embodiment, the condition is pruritis.

Also provided herein is an antigen-binding molecule, or vector, as described herein, for use in treating, inhibiting or ameliorating a condition associated with increased expression and/or increased activity of IL-31 in a subject.

Also provided herein is the use of the IL-31-binding molecules, or vector, as described herein, in the manufacture of a medicament for treating, inhibiting or ameliorating a condition associated with an abnormal (e.g., increased) level and/or abnormal (e.g., increased) activity of IL-31 in a subject in need thereof, as described herein.

Also disclosed herein is a method of treating or preventing a condition caused by, associated with, or resulting from, an increased expression of IL-31 or increased sensitivity to IL-31 in a subject in need thereof, the method comprising the step of administering the IL-31-binding molecule, or vector, as described herein, to a canine subject in need thereof.

Also disclosed herein is the IL-31-binding molecule, or the vector, or the pharmaceutical composition, as described herein, for use in the treatment of a condition caused by, associated with, or resulting from, an increased expression of IL-31 or increased sensitivity to IL-31 in a subject.

The present disclosure also extends to the use of the IL-31-binding molecule, or the vector, as described herein, in the manufacture of a medicament for the treatment of a condition caused by, associated with, or resulting from, an increased expression of IL-31 or increased sensitivity to IL-31 in a subject.

The present disclosure also extends to a method for the treatment or prevention of a tumour induced to proliferate by IL-31 and conditions associated therewith, the method comprising administering the IL-31-binding molecule, or the vector, or the pharmaceutical composition, as described herein, to a subject in need thereof.

Also provided herein is the IL-31-binding molecule, or the vector, or the pharmaceutical composition, as described herein, for use in the treatment or prevention of a tumour induced to proliferate by IL-31 and conditions associated therewith, in a subject in need thereof.

The present disclosure also extends to the use of the IL-31-binding molecule, or the vector, as described herein, in the manufacture of a medicament for the treatment or prevention of a tumour induced to proliferate by IL-31 and conditions associated therewith, in a subject in need thereof.

The present disclosure also extends to a kit comprising the IL-31-binding molecule, or the vector, or the pharmaceutical composition, as described herein.

Also disclosed herein is the use of the IL-31-binding molecule, or the vector, as described herein, for detecting IL-31 in a sample.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1: Generation of Mouse Monoclonal Antibodies Recognizing Canine Interleukin 31 (IL-31)

Recombinant canine IL-31 (ProSpec-Tany, Israel) produced in Sf9 Baculovirus cells comprising 142 amino acids (amino acid residues 24-159 of Accession no. NP_001159386; SEQ ID NO:1) fused to a 6 amino acid His-Tag at C-terminus was used as an immunogen to generate antibodies in Balb/c mice.

### Canine IL-31 Protein

(SEQ ID NO: 1)
SHMAPTHQLPPSDVRKIILELQPLSRGLLEDYQKKETGVP
ESNRTLLLCLTSDSQPPRLNSSAILPYFRAIRPLSDKNII
DKIIEQLDKLKFQHEPETEISVPADTFECKSFILTILQQF
SACLESVFKSLNSGPQ

Antibody titres from immunized mice were determined using an enzyme linked immunosorbent assay (ELISA). Canine IL-31 (100 ng/well) was immobilized to polystyrene microplates and used as a capture antigen and blocked with PBS/0.05% Tween 20/1% BSA. Coated wells were incubated for 1 hour at room temperature with monoclonal antibody (mAb) diluted in PBS/0.05% Tween 20/1% BSA (100 μl/well). mAb concentrations ranging from 1000 ng/ml to 1.37 ng/ml were used to establish a standard curve. After washing, the plates were incubated with a 1/10,000 dilution Alkaline Phosphatase-conjugated AffiniPure Goat anti-Mouse IgG (subclasses 1+2a+2b+3), Fcg Fragment Specific (Jackson Immuno Research Laboratories, Inc) or an Alkaline Phosphatase-conjugated Rabbit Anti-Dog IgG (whole molecule; Sigma) in PBS/0.05% Tween 20/1% BSA. Plates were washed with PBS/0.05% Tween 20 and developed by the addition of 3,3′,5,5′-Tetramethylbenzidine (TMB) substrate. Development was stopped by the addition of 100 μl 2M H2504 and absorbance of each well was determined at an optical density (OD) of 450 nm.

The spleens of 3 mice that were immunized with canine IL-31 protein (SEQ ID NO:1) were isolated and individually used for hybridoma generation using standard technologies (see, for example, Albitar (2007; Methods in Molecular Biology; Monoclonal Antibodies Volume 378∥Development and Characterization of Mouse Hybridomas, 10.1007/978-1-59745-323-3 (Chapter 1). Following fusion, hybridomas were plated out into multi-well plates. 6000 wells were screened for binding to canine IL-31 using a microarray ELISA format.

Of the candidates identified during the screen, one hybridoma demonstrated good binding to canine IL-31 and inhibition of canine IL-31 activity. This hybridoma was subsequently cloned and sequenced (clone designation RA4.D2).

The sequences of the heavy chain variable region (VH) and the light chain variable region (VL) of the subclones for RA4.D2 were obtained by PCR amplification of the VH and VL regions from cDNA obtained from the hybridomas. Analysis of the VH and VL gene sequences was performed using the IMGT/V-Questprogram, (The International Immunogenetics Information System; http://www.imgt.org/IMGT_vquest/vquest).

To produce a chimeric antibody, chRA4, the heavy chain variable sequence of the murine monoclonal antibody RA4 was fused to the constant region of the canine immunoglobulin gamma heavy chain A of SEQ ID NO:2 (corresponding to amino acid positions 138-468 of GenBank: AAL35301), and the light chain variable sequence was fused to the constant region corresponding to the IGKC*01 allele from IMGT (accession number IMGT000067) as shown below:

Canine Heavy Chain Constant Region
(SEQ ID NO: 2)
ASTTAPSVFPLAPSCGSTSGSTVALACLVSGYFPEPVTVS
WNSGSLTSGVHTFPSVLQSSGLHSLSSMVTVPSSRWPSET
FTCNVVHPASNTKVDKPVFNECRCTDTPPCPVPEPLGGPS
VLIFPPKPKDILRITRTPEVTCVVLDLGREDPEVQISWFV
DGKEVHTAKTQSREQQFNGTYRVVSVLPIEHQDWLTGKEF
KCRVNHIDLPSPIERTISKARGRAHKPSVYVLPPSPKELS
SSDTVSITCLIKDFYPPDIDVEWQSNGQQEPERKHRMTPP
QLDEDGSYFLYSKLSVDKSRWQQGDPFTCAVMHETLQNHY
TDLSLSHSPGK
Canine kappa Light Chain Constant Region
(SEQ ID NO: 80)
RNDAQPAVYLFQPSPDQLHTGSASVVCLLNSFYPKDINVK
WKVDGVIQDTGIQESVTEQDKDSTYSLSSTLTMSSTEYLS
HELYSCEITHKSLPSTLIKSFQRSECQRVD

TABLE 1A
CDR sequences of the Heavy
Chain (HC) and Light Chain
(LC) variable regions of monoclonal
antibody RD4.D2 (IMGT defined)

| | HC_CDR1 | HC_CDR2 | HC_CDR3 |
|---|---|---|---|
| RA4.D2 | GYTFTDYS | INTETGKT | ASGRPDY |
| | (SEQ ID | (SEQ ID | (SEQ ID |
| | NO: 6) | NO: 7) | NO: 8) |
| | LC_CDR1 | LC_CDR2 | LC_CDR3 |
| RA4.D2 | ESVEYYGTSL | AAS | QQSRKVPYT |
| | (SEQ ID | (SEQ ID | (SEQ ID |
| | NO: 9) | NO: 10) | NO: 11) |

TABLE 1B
CDR sequences of the Heavy Chain (HC
and Light Chain (LC) variable
regions of monoclonal antibody RD4.D2
(identified using Kabat numbering)

| | HC_CDR1 | HC_CDR2 | HC_CDR3 |
|---|---|---|---|
| RA4.D2 | DYSMH | WINTETGKT | GRPDY |
| | (SEQ ID | IHAEDFKG | (SEQ ID |
| | NO: 93) | (SEQ ID | NO: 95) |
| | | NO: 94) | |
| | LC_CDR1 | LC_CDR2 | LC_CDR3 |
| RA4.D2 | RASESVEY | AASGVES | QQSRKVPYT |
| | YGTSLMQ | (SEQ ID | (SEQ ID |
| | (SEQ ID | NO: 97) | NO: 98) |
| | NO: 96) | | |

Amino acid sequences of the heavy chain (VH) and light chain (VL) variable regions of the murine monoclonal antibody RA4.D2 (CDR sequences (IMGT-identified) are underlined)

RA4_VH
(SEQ ID NO: 12)
QIQLVQSGPELKKPGETVKISCKASGYTFTDYSMHWVKQA
PGKGLRWMGWINTETGKTIHAEDFKGRVAFSLETSASTAY
LQINNLKNEDTATYFCASGRPDYWGQGTTLTVSS
RA4_VL
(SEQ ID NO: 13)
DIVLSQSPASLAVSLGQRATISCRASESVEYYGTSLMQWY
QQKPGQPPKLLIYAASGVESGVPARFSGSGSGTDFSLIIH
PVEEDDIAMYFCQQSRKVPYTFGGGTKLEIKRADAAPTVS

### Nucleotide Sequences Encoding the Heavy Chain (VH) and Light Chain (VL) Variable Regions of the Murine Monoclonal Antibody RA4.D2

RA4_Heavy-chain variable region (VH)
(SEQ ID NO: 14)
CAGATCCAGTTGGTGCAGTCTGGACCTGAGCTGAAGAAGC
CAGGAGAGACAGTCAAGATCTCCTGCAAGGCTTCTGGTTA
TACCTTCACAGACTATTCAATGCACTGGGTGAAGCAGGCT
CCAGGAAAGGGTTTAAGGTGGATGGGCTGGATAAACACTG
AGACTGGTAAGACAATACACGCAGAAGACTTCAAGGGACG
GGTTGCCTTCTCTTTGGAAACCTCTGCCAGCACTGCCTAT
TTGCAGATCAACAACCTCAAAAATGAGGACACGGCTACAT
ATTTCTGTGCTAGCGGGCGGCCTGACTACTGGGGCCAAGG
CACCACTCTCACAGTCTCCTCA
RA4_Light-chain variable region (VL);
(kappa light chain)
(SEQ ID NO: 15)
GACATTGTGCTCTCCCAATCTCCAGCTTCTTTGGCTGTGT
CTCTAGGGCAGAGAGCCACCATCTCCTGCAGAGCCAGTGA
AAGTGTTGAATATTATGGCACAAGTTTGATGCAGTGGTAC
CAACAGAAGCCAGGACAGCCACCCAAACTCCTCATCTATG
CTGCATCCGGCGTAGAATCTGGGGTCCCTGCCAGGTTTAG
TGGCAGTGGGTCTGGGACAGACTTCAGCCTCATCATCCAT
CCTGTGGAGGAGGATGATATTGCAATGTATTTCTGTCAGC
AAAGTAGGAAGGTTCCGTACACGTTCGGAGGGGGGACCAA
ACTGGAAATAAAACGG

### Amino Acid Sequences of the Heavy Chain (HC) and Light Chain (LC) Regions of the Chimeric RA4.D2 Antibody

chRA4_HC
(SEQ ID NO: 17)
QIQLVQSGPELKKPGETVKISCKASGYTFTDYSMHWVKQA
PGKGLRWMGWINTETGKTIHAEDFKGRVAFSLETSASTAY
LQINNLKNEDTATYFCASGRPDYWGQGTTLTVSSASTTAP
SVFPLAPSCGSTSGSTVALACLVSGYFPEPVTVSWNSGSL
TSGVHTFPSVLQSSGLHSLSSMVTVPSSRWPSETFTCNVV
HPASNTKVDKPVFNECRCTDTPPCPVPEPLGGPSVLIFPP
KPKDILRITRTPEVTCVVLDLGREDPEVQISWFVDGKEVH
TAKTQSREQQFNGTYRVVSVLPIEHQDWLTGKEFKCRVNH
IDLPSPIERTISKARGRAHKPSVYVLPPSPKELSSSDTVS
ITCLIKDFYPPDIDVEWQSNGQQEPERKHRMTPPQLDEDG
SYFLYSKLSVDKSRWQQGDPFTCAVMHETLQNHYTDLSLS
HSPGK
chRA4_LC
(SEQ ID NO: 16)
DIVLSQSPASLAVSLGQRATISCRASESVEYYGTSLMQWY
QQKPGQPPKLLIYAASGVESGVPARFSGSGSGTDFSLIIH
PVEEDDIAMYFCQQSRKVPYTFGGGTKLEIKRNDAQPAVY
LFQPSPDQLHTGSASVVCLLNSFYPKDINVKWKVDGVIQD
TGIQESVTEQDKDSTYSLSSTLTMSSTEYLSHELYSCEIT
HKSLPSTLIKSFORSECQRVD

To produce a caninized RA4 monoclonal antibody, the framework regions of the heavy and light chain variable sequences of the murine monoclonal antibody RA4 were substituted for canine heavy and light chain framework regions. Canine antibody sequences were searched for comparison to the murine heavy and light chain variable domains using BLAST search algorithms, and candidate canine variable domains selected from the top 200 BLAST results. The top candidates were selected based on a combination of framework homology, maintaining key framework residues and canonical loop structure.

The amino acid sequences of the caninized RA4 heavy and light chain variable regions are set out below:

RA4 Caninized Heavy Chain
VHC5
(SEQ ID NO: 99)
EVQLVQSGPEVKKPGESVKVSCKTSGYTFTDYSMHWVRQA
PGKGLRWMGWINTETGKTIHAEDFKGRVTLTADTSTSTAY
MQLSSLRAEDIAVYFCASGRPDYWGQGTTLTVSS
VHC6
(SEQ ID NO: 100)
QVQLVQSGAEVKKPGESVKVSCKTSGYTFTDYSMHWVRQA
PGKGLDWMGWINTETGKTIHAEDFKGRVTLTADTSTSTAY
MELSSLRAGDIAVYFCASGRPDYWGQGTTLTVSS
RA4 Caninized Light Chain
VLC6
(SEQ ID NO: 101)
EIVLTQSPASLSLSQGEKVTITCRASESVEYYGTSLMQWY
QQKPGQAPKLLIYAASGVESGVPSRFSGSGSGTDFSFTIS
SLEPEDVAVYFCQQSRKVPYTFGGGTKLEIK

TABLE 2
Amino acid sequences of the heavy chain
(VH1) and the light chain (VL)
framework regions (FR1-4) of a
caninized IL-31-binding molecule:

| | Caninized VH Framework Region |
|---|---|
| | (FR) Sequences |
| VH1 | FR1 QVQLQESGPGLVKPSQTLSLTCTVS |
| | (SEQ ID NO: 18) |
| | FR2 WVRQRTGRGLEWMG |
| | (SEQ ID NO: 19) |
| | FR3 RLSITRDTAKSQVSLQMSSMTTEDTATYYCAR |
| | (SEQ ID NO: 20) |
| | FR4 WGQGTSVTVSS |
| | (SEQ ID NO: 21) |
| VH2 | FR1 QVQLQESGPGLVKPSQTLSLTCTVS |
| | (SEQ ID NO: 22) |
| | FR2 WVRQRPGRGLEWMG |
| | (SEQ ID NO: 23) |
| | FR3 RLSITRDTAKSQVSLQMSSMTTEDTATYYCAR |
| | (SEQ ID NO: 24) |
| | FR4 WGQGTLVTVSS |
| | (SEQ ID NO: 25) |
| VH3 | FR1 QVQLQESGPGLVKPSQTLSLTCTVS |
| | (SEQ ID NO: 26) |
| | FR2 WVRQRPGRGLEWMG |
| | (SEQ ID NO: 27) |
| | FR3 RISITRDTAKSQVSLQLSSMTTEDTATYYCAR |
| | (SEQ ID NO: 28) |
| | FR4 WGQGTLVTVSS |
| | (SEQ ID NO: 29) |
| VH4 | FR1 EVTLQESGPGLVKPSQTLSLTCTVS |
| | (SEQ ID NO: 30) |
| | FR2 WVRQRPGRGLEWMG |
| | (SEQ ID NO: 31) |
| | FR3 RISITRDTAKNQVSLQLSSMTTEDTATYYCAR |
| | (SEQ ID NO: 32) |
| | FR4 WGQGTLVTVSS |
| | (SEQ ID NO: 33) |
| VH5 | FR1 EVTLQESGPGLVKPSQTLSLTCVVS |
| | (SEQ ID NO: 34) |
| | FR2 WVRQRPGRGLEWMG |
| | (SEQ ID NO: 35) |
| | FR3 RISITRDTAKNQVSLQLSSMTTEDTAVYYCAR |
| | (SEQ ID NO: 36) |
| | FR4 WGQGTLVTVSS |
| | (SEQ ID NO: 37) |
| VH6 | FR1 EVQLVQSGPEVKKPGESVKVSCKTS |
| | (SEQ ID NO: 81) |
| | FR2 WVRQAPGKGLRWMG |
| | (SEQ ID NO: 82) |
| | FR3 RVTLTADTSTSTAYMQLSSLRAEDIAVYFC |
| | (SEQ ID NO: 83) |
| | FR4 WGQGTTLTVSS |
| | (SEQ ID NO: 84) |
| VH7 | FR1 QVQLVQSGAEVKKPGESVKVSCKTS |
| | (SEQ ID NO: 85) |
| | FR2 WVRQAPGKGLDWMG |
| | (SEQ ID NO: 86) |
| | FR3 RVTLTADTSTSTAYMELSSLRAGDIAVYFC |
| | (SEQ ID NO: 87) |
| | FR4 WGQGTTLTVSS |
| | (SEQ ID NO: 88) |
| VL1 | FR1 DIQMTQSPASLSLSQEEKVTITC |
| | (SEQ ID NO: 38) |
| | FR2 WYQQKPGQAPKLLIY |
| | (SEQ ID NO: 39) |
| | FR3 GVPSRFSGSGSGTDYSFTISSLESEDVASYFC |
| | (SEQ ID NO: 40) |
| | FR4 FGAGTKVELK |
| | (SEQ ID NO: 41) |
| VL2 | FR1: DIQMTQSPASLSLSQEEKVTITC |
| | (SEQ ID NO: 42) |
| | FR2: WYQQKPGQAPKLLIY |
| | (SEQ ID NO: 43) |
| | FR3: GVPSRFSGSGSGTDYSFTISSLEPEDVASYFC |
| | (SEQ ID NO: 44) |
| | FR4: FGAGTKVELK |
| | (SEQ ID NO: 45) |
| VL3 | FR1: EIVMTQSPASLSLSQEEKVTITC |
| | (SEQ ID NO: 46) |
| | FR2: WYQQKPGQAPKLLIY |
| | (SEQ ID NO: 47) |
| | FR3: GVPSRFSGSGSGTDYSFTISSLEPEDVASYFC |
| | (SEQ ID NO: 48) |
| | FR4: FGAGTKVELK |
| | (SEQ ID NO: 49) |
| VL4 | FR1: EIVMTQSPASLSLSQEEKVTITC |
| | (SEQ ID NO: 50) |
| | FR2: WYQQKPGQAPKLLIY |
| | (SEQ ID NO: 51) |
| | FR3: GVPSRFSGSGSGTDYSFTISSLEPEDVAVYFC |
| | (SEQ ID NO: 52) |
| | FR4: FGAGTKVELK |
| | (SEQ ID NO: 53) |
| VL5 | FR1: EIVLTQSPASLSLSQGEKVTITC |
| | (SEQ ID NO: 89) |
| | FR2: WYQQKPGQAPKLLIY |
| | (SEQ ID NO: 90) |
| | FR3: GVPSRFSGSGSGTDFSFTISSLEPEDVAVYFC |
| | (SEQ ID NO: 91) |
| | FR4: FGGGTKLEIK |
| | (SEQ ID NO: 92) |

To produce a felinized RA4 monoclonal antibody, the framework regions of the heavy and light chain variable sequences of the murine monoclonal antibody RA4 will be substituted for feline heavy and light chain framework region sequences. Feline antibody sequences will be searched for comparison to the murine heavy and light chain variable domains using BLAST search algorithms, and candidate feline variable domains will be selected from the top 200 BLAST results. The top candidates will be selected based on a combination of framework homology, maintaining key framework residues and canonical loop structure.

TABLE 3
Illustrative examples of amino acid
sequences of heavy chain (VH) and the
light chain (VL) framework regions
(FR1-4) of a felinized IL-31-binding
molecule:

VH Framework Region Sequences
FR1: QVQLMESGADLVQPSESLRLTCVAS (SEQ ID NO: 54)
FR2: WVRQAPGKGLEWMG (SEQ ID NO: 55)
FR3: RLTITRDTSKNTVFLQMHSLQSEDTATYYCAR
(SEQ ID NO: 56)
FR4: WGQGTTVTVSA (SEQ ID NO: 57)
VL Framework Region Sequences
FR1: DIEMTQSPLSLSATPGETVSISC (SEQ ID NO: 58)
FR2: WYLQKPGRSPRLLIY (SEQ ID NO: 59)
FR3: GVPDRFSGSGSGTDFTLKISRVQTEDVGVYFC
(SEQ ID NO: 60)
FR4: FGQGTKLELK (SEQ ID NO: 61)

### Example 2: Determination of Binding of Monoclonal Antibody RA4 to Canine IL-31, Feline IL-31 and Canine IL-31 Mutants

To evaluate if the murine monoclonal antibody RA4 bound to the region of canine IL-31 targeted by previously published anti-IL-31 antibodies, M14 (as described in U.S. Pat. No. 10,093,731 B2) and mAb 34D03 (as described in U.S. Pat. No. 8,790,651 B2), two mutant canine IL-31 proteins were generated with alanine substitutions introduced at (i) amino acid positions 76, 77, 80, 81 and 84 of SEQ ID NO:1 (canine IL-31 Mutant 1; SEQ ID NO:62) and (ii) amino acid positions 13, 15, 20 and 26 of SEQ ID NO:1 (canine IL-31 Mutant 2; SEQ ID NO:63). The amino acid positions of canine IL-31 Mutants 1 and 2 that were targeted for alanine substitution were previously determined to be involved in the binding of 34D03 and M14 to canine IL-31, respectively.

The amino acid sequences of canine IL-31 Mutants 1 and 2 and feline IL-31 proteins are provided below:

Canine IL-31 Mutant 1 (the alanine substitutions
are identified by bold and underlined
text)
(SEQ ID NO: 62)
SHMAPTHQLPPSDVRKIILELQPLSRGLLEDYQKKETGVPESNRTL
LLCLTSDSQPPRLNSSAILPYFRAIRPLSAANIAAKIAEQLDKLKF
QHEPETEISVPADTFECKSFILTILQQFSACLESVFKSLNSGPQ
Canine IL-31 Mutant 2 (alanine substitutions are
identified by bold and underlined text)
(SEQ ID NO: 63)
SHMAPTHQLPPSAVAKIILALQPLSAGLLEDYQKKETGVPESNRTL
LLCLTSDSQPPRLNSSAILPYFRAIRPLSDKNIIDKIIEQLDKLKF
QHEPETEISVPADTFECKSFILTILQQFSACLESVFKSLNSGPQ
Feline IL-31
(SEQ ID NO: 64)
SHMAPAHRLQPSDVRKIILELRPMSKGLLQDYLKKEIGLPESNHSS
LPCLSSDSQLPHINGSAILPYFRAIRPLSDKNTIDKIIEQLDKLKF
QREPEAKVSMPADNFERKNFILAVLQQFSACLEHVLQSLNSGPQ

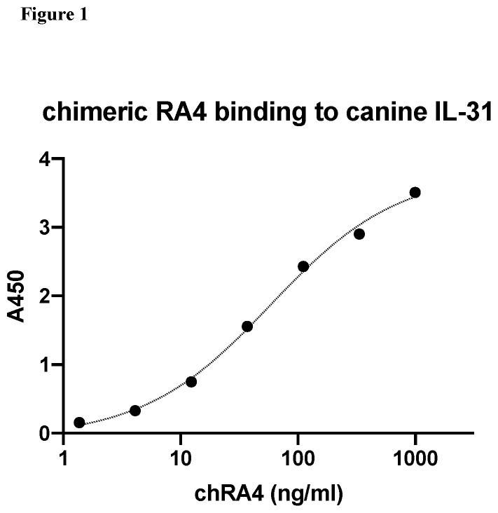

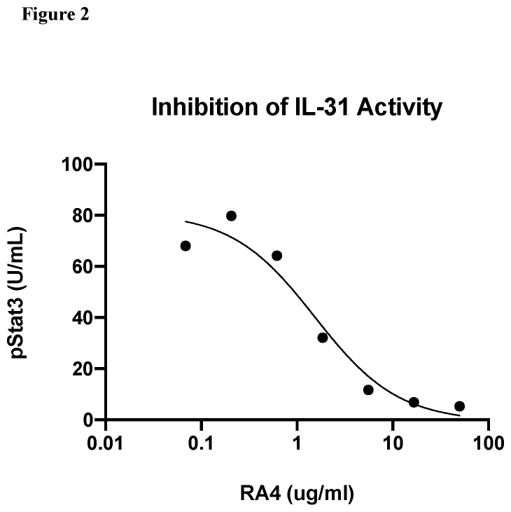

Purified murine RA4 and the recombinant chimeric RA4 mAb were analysed for binding to canine IL-31 by ELISA, as described above. As shown in FIG. 1, the chimeric antibody RA4 (chRA4) bound to canine IL-31 in a dose-dependent manner, as determined by ELISA. Consistent with these binding data, chRA4 was shown to neutralise canine IL-31 activity in vitro, as evidenced by inhibition of canine IL-31-induced STAT-3 phosphorylation in canine DH82 cells (FIG. 2). The ability of RA4 mAb to neutralise IL-31-mediated pSTAT3-signalling in a canine monocyte cell line was assessed as follows: canine DH-82 cells were pre-treated for 24 hours with canine gamma interferon at 10 ng/mL and then serum starved for 2 hours. Cell supernatants were incubated with canine IL-31 (160 ng/mL) for 60 minutes. The RA4/IL-31 mix was then added to cells for 5 min and STAT phosphorylation was evaluated using a pSTAT3 ELISA (STAT-3 (Phospho) [pY705] Multispecies ELISA Kit, Invitrogen).

To evaluate if the RA4 mAb bound to the region of canine IL-31 targeted by previously published anti-IL-31 antibodies M14 and 34D03, two mutant canine IL-31 proteins were generated with alanine substitutions introduced at (i) amino acid positions 76, 77, 80, 81 and 84 of SEQ ID NO:1 (canine IL-31 Mutant 1; SEQ ID NO:62) and (ii) amino acid positions 13, 15, 20 and 26 of SEQ ID NO:1 (canine IL-31 Mutant 2; SEQ ID NO:63).

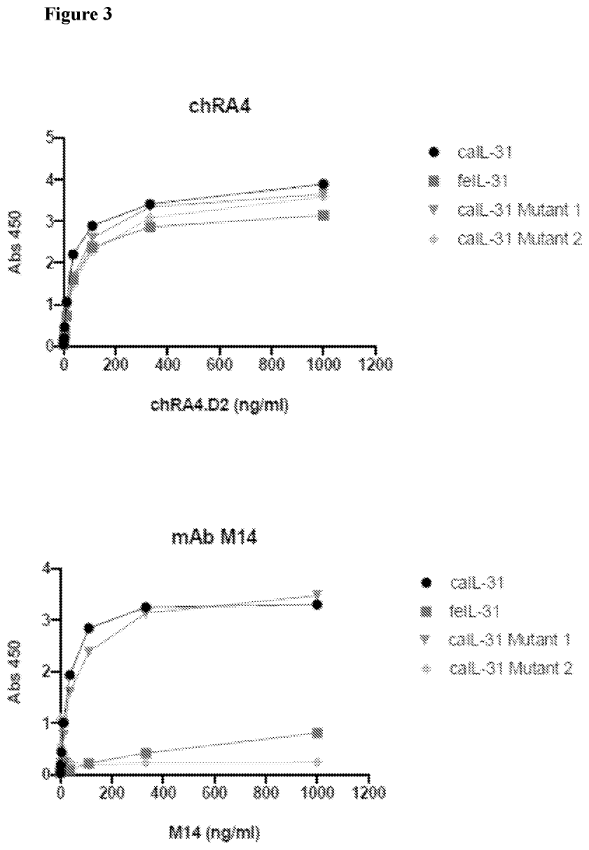

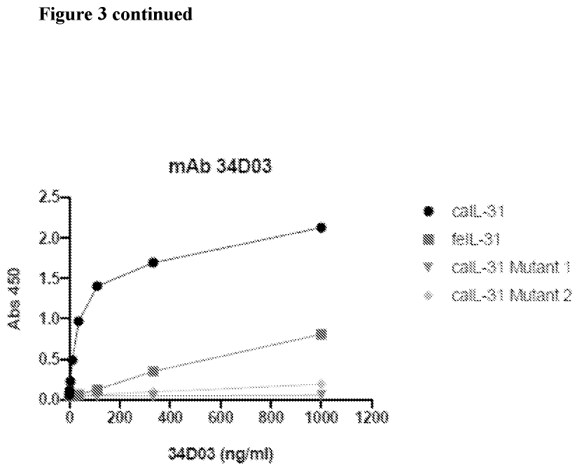

As shown in FIG. 3, RA4 bound to canine IL-31, feline IL-31 (SEQ ID NO: 64) and to both canine IL-31 mutant proteins, Mutant 1 (SEQ ID NO:62) and Mutant 2 (SEQ ID NO:63). M14 bound to canine IL-31 and to canine IL-31 Mutant 1, but did not show significant binding to canine IL-31 Mutant 2. M14 shows minimal binding to feline IL-31. 34D03 also bound to canine IL-31 and to a lesser extent, feline IL-31, but did not show significant binding to either Mutant 1 or Mutant 2. RA4 did not bind to human IL-31 in these studies.

### Example 3: Identification of the Binding Epitope on Canine Interleukin 31 (calL-31) for Anti-caIL-31 mAb Clone RA4

As noted in Example 2, above, the murine RA4 mAb does not bind to critical residues involved in the binding of anti-canine IL-31 mAbs M14 and 34D03.

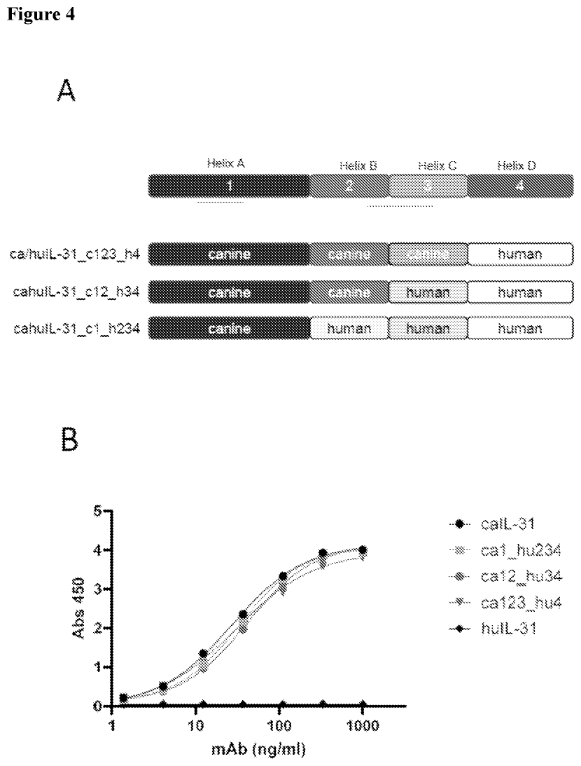

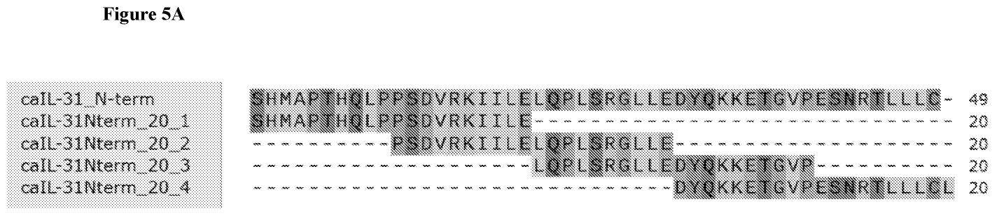

To define the region of canine IL-31 that binds RA4, a number of chimeric and truncated canine IL-31 peptides were generated, as set out below (see also FIG. 4A). The human IL-31-derived amino acid sequences are identified by underlined text):

Canine IL-31
(SEQ ID NO: 1)
SHMAPTHQLPPSDVRKIILELQPLSRGLLEDYQKKETGVP
ESNRTLLLCLTSDSQPPRLNSSAILPYFRAIRPLSDKNII
DKIIEQLDKLKFQHEPETEISVPADTFECKSFILTILQQF
SACLESVFKSLNSGPQ
Canine/Human chIL-31_c123_h4
(SEQ ID NO: 65)
SHMAPTHQLPPSDVRKIILELQPLSRGLLEDYQKKETGVP
ESNRTLLLCLTSDSQPPRLNSSAILPYFRAIRPLSDKNII
DKIIEQLDKLKFQHEPETNISVPTDTHECKRFILTISQQF
SECMDLALKSLTSGAQQATT
Canine/Human chIL-31_c12_h34
(SEQ ID NO: 66)
SHMAPTHQLPPSDVRKIILELQPLSRGLLEDYQKKETGVP
ESNRTLLLCLTSDSQPPRLNSSAILPYFRAIRPLSDKSVI
DEIIEHLDKLIFQDAPETNISVPTDTHECKRFILTISQQF
SECMDLALKSLTSGAQQATT
Canine/Human chIL-31_c1_h234
(SEQ ID NO: 67)
SHMAPTHQLPPSDVRKIILELQPLSRGLLEDYQKKETGVP
ESNRTLLLCLSPDAQPPNNIHSPAIRAYLKTIRQLDNKSV
IDEIIEHLDKLIFQDAPETNISVPTDTHECKRFILTISQQ
FSECMDLALKSLTSGAQQATT

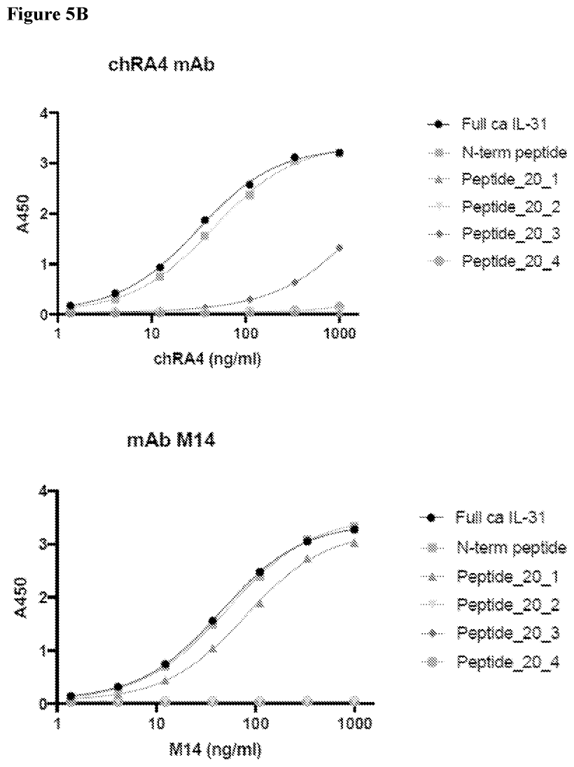

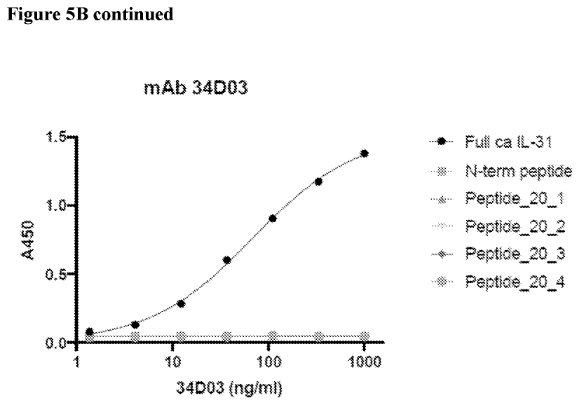

The amino acid sequences of the canine IL-31 proteins identified in FIG. 5 are as follows:

Canine IL-31_N-term
(SEQ ID NO: 68)
SHMAPTHQLPPSDVRKIILELQPLS
RGLLEDYQKKETGVPESNRTLLLC
Canine IL-31_N-term_20_1
(SEQ ID NO: 69)
SHMAPTHQLPPSDVRKIILE
Canine IL-31_N-term_20_2
(SEQ ID NO: 70)
PSDVRKIILELQPLSRGLLE
Canine IL-31_N-term_20_3
(SEQ ID NO: 71)
LQPLSRGLLEDYQKKETGVP
Canine IL-31_N-term_20_4
(SEQ ID NO: 72)
DYQKKETGVPESNRTLLLCL

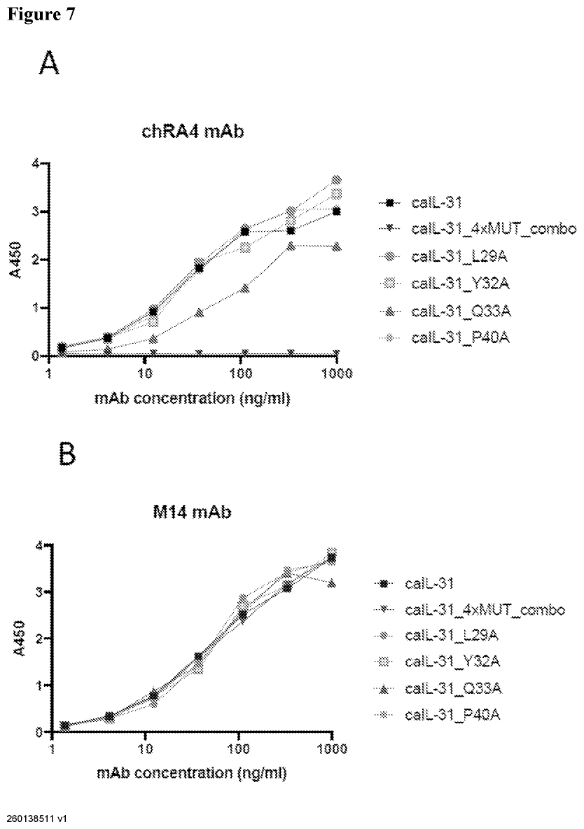

The amino acid sequences of the canine IL-31 proteins identified in FIG. 7 are as follows:

Canine IL-31
(SEQ ID NO: 73)
MLSHTGPSRFALFLLCSMETLLSSHMAPTHQLPPSDVRKI
ILELQPLSRGLLEDYQKKETGVPESNRTLLLCLTSDSQPP
RLNSSAILPYFRAIRPLSDKNIIDKIIEQLDKLKFQHEPE
TEISVPADTFECKSFILTILQQFSACLESVFKSLNSGPQH
HHHHH
Canine IL-31_4xMUT_combo
(SEQ ID NO: 74)
MLSHTGPSRFALFLLCSMETLLSSHMAPTHQLPPSDVRKI
ILELQPLSRGLAEDAAKKETGVAESNRTLLLCLTSDSQPP
RLNSSAILPYFRAIRPLSDKNIIDKIIEQLDKLKFQHEPE
TEISVPADTFECKSFILTILQQFSACLESVFKSLNSGPQH
HHHHH
Canine IL-31_L29A
(SEQ ID NO: 75)
MLSHTGPSRFALFLLCSMETLLSSHMAPTHQLPPSDVRKI
ILELQPLSRGLAEDYQKKETGVPESNRTLLLCLTSDSQPP
RLNSSAILPYFRAIRPLSDKNIIDKIIEQLDKLKFQHEPE
TEISVPADTFECKSFILTILQQFSACLESVFKSLNSGPQH
HHHHH
Canine IL-31_Y32A
(SEQ ID NO: 76)
MLSHTGPSRFALFLLCSMETLLSSHMAPTHQLPPSDVRKI
ILELQPLSRGLLEDAQKKETGVPESNRTLLLCLTSDSQPP
RLNSSAILPYFRAIRPLSDKNIIDKIIEQLDKLKFQHEPE
TEISVPADTFECKSFILTILQQFSACLESVFKSLNSGPQH
HHHHH
Canine IL-31_Q33A
(SEQ ID NO: 77)
MLSHTGPSRFALFLLCSMETLLSSHMAPTHQLPPSDVRKI
ILELQPLSRGLLEDYAKKETGVPESNRTLLLCLTSDSQPP
RLNSSAILPYFRAIRPLSDKNIIDKIIEQLDKLKFQHEPE
TEISVP ADTFECKSFILTILQQFSACLESVFKSLNSGPQ
HHHHHH
Canine IL-31_P40A
(SEQ ID NO: 78)
MLSHTGPSRFALFLLCSMETLLSSHMAPTHQLPPSDVRKI
ILELQPLSRGLLEDYQKKETGVAESNRTLLLCLTSDSQPP
RLNSSAILPYFRAIRPLSDKNIIDKIIEQLDKLKFQHEPE
TEISVPADTFECKSFILTILQQFSACLESVFKSLNSGPQH
HHHHH

Binding to the various IL-31 proteins and peptides was assessed by ELISA. IL-31 proteins or peptides (100 ng/well) were immobilized to polystyrene microplates as capture antigen. Wells were blocked with PBS/0.05% Tween 20/1% BSA. Coated wells were incubated for 1 h at room temperature with anti-IL-31 mAbs diluted in PBS/0.05% Tween 20/1% BSA (100 μl/well). After washing, the plates were incubated with a 1/10,000 dilution Alkaline Phosphatase-conjugated Rabbit Anti-Dog IgG (whole molecule) (Sigma) in PBS/0.05% Tween 20/1% BSA. Plates were washed with PBS/0.05% Tween 20 and developed by the addition of TMB substrate. Development was stopped by the addition of 100 ul ELISA stop Solution (Invitrogen) and absorbance of each well was determined at an optical density (OD) of 450 nm.

As shown in FIG. 4B, the RA4 mAb binds to the chimeric canine/human IL-31 proteins that contain the first 49 amino acids of canine IL-31 (SEQ ID NO:1) which incorporates the helix A of IL-31. Replacement of the canine sequence for helices B, C and D with the human sequence did not abrogate binding, demonstrating that the epitope of RA4 mAb resides in the N-terminal region of canine IL-31.

Overlapping peptides of 20 amino acids were generated (FIG. 5A) to further define the epitope of RA4 mAb. As shown by the ELISA data in FIG. 5B, RA4 mAb binds to the peptide containing the first 50 amino acids of canine IL-31 and to Peptide 3, which contains amino acid residues 30-40 of mature canine IL-31. The epitope that is bound by RA4 mAb is different to the epitopes targeted by anti-canine IL-31 antibodies M14 and 34D03. 34D03 does not appear to bind to the N-terminal region of canine IL-31, consistent with the data in FIG. 3, which suggest that the epitope bound by 34D03 is found in Helices B/C. In contrast, M14 bound only to Peptide 1, comprising amino acid residues 1-20 of canine IL-31. These data show that the RA4 mAb binds to a unique epitope of canine IL-31, not previously described for M14 and 34D03.

An alanine-scanning peptide library of the region encompassing amino acid residues 18-42 of SEQ ID NO:1 was generated (IILELQPLSRGLLEDYQKKETGVPES; SEQ ID NO:79), consisting of 25 peptides, each with a single alanine mutation at a different residue. Binding of the RA4 mAb to each of the peptides was determined by ELISA, as detailed above.

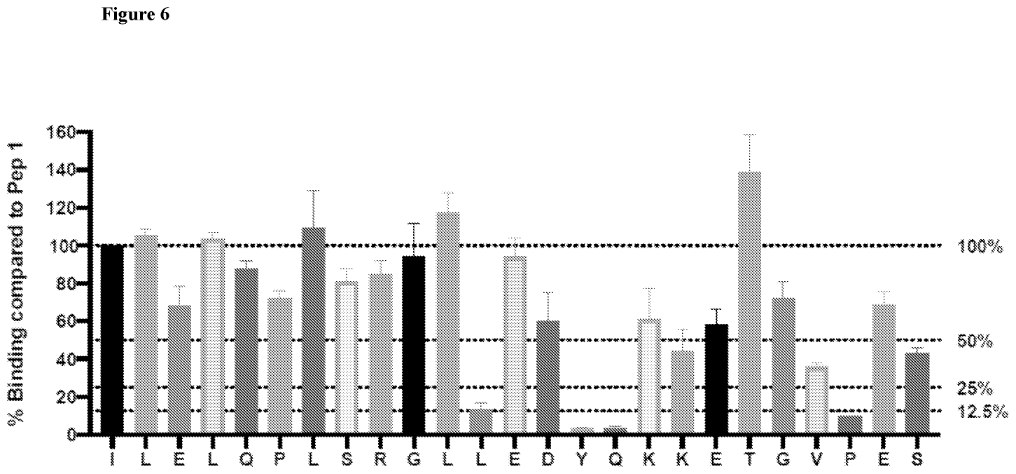

As shown in FIG. 6, where binding to each of the peptides is shown as a percentage of binding to the first peptide in the series, the data demonstrate 4 key residues that, when substituted for alanine, severely impacted the binding of RA4 to canine IL-31. Alanine mutations corresponding to L29A, Y32A, Q33A and P40A all reduced binding to canine IL-31 peptide to less than 15% of the first peptide. A further 3 residues were also shown to be important to binding to canine IL-31 that, when substituted for an alanine residue, reduced binding to less than 50% (K35A, V39A and S42A).

Confirmation of the unique epitope targeted by RA4 mAb was performed by assessing the binding of RA4 mAb to full length canine IL-31 proteins that contained each of the 4 key residues identified above, substituted for alanine, and a version with all 4 residues substituted for alanine in combination. Histidine-tagged proteins were generated in mammalian cells following transient transfection and were purified via nickel affinity chromatography. Binding of RA4 mAb and M14 mAb to each of the proteins was determined by ELISA, as detailed above. As shown in FIG. 7, the chimeric RA4 mAb was able to bind all proteins with a single amino acid alanine substitution when comparable to binding to the wild-type canine IL-31 protein (FIG. 7A). When all 4 residues (L29, Y32, Q33, and P40) were substituted for alanine in combination, no binding to canine IL-31 was detected. In contrast, M14 mAb bound equally well to all mutant proteins and wild-type canine IL-31 protein. These data show that an epitope comprising amino acid residues L29, Y32, Q33 and P40 of canine 11-31 (SEQ ID NO:1) is unique to the RA4 mAb.

1. An antigen-binding molecule that specifically binds to interleukin-31 (IL-31), wherein the antigen-binding molecule comprises an immunoglobulin heavy chain variable domain (VH) and an immunoglobulin light chain variable domain (VL), wherein the VH comprises a complementarity determining region 1 (VH CDR1) comprising the amino acid sequence of SEQ ID NO: 6 or an amino acid sequence having at least 80% sequence identity thereto, a VH CDR2 comprising the amino acid sequence of SEQ ID NO: 7 or an amino acid sequence having at least 80% sequence identity thereto and a VH CDR3 comprising the amino acid sequence of SEQ ID NO: 8 or an amino acid sequence having at least 80% sequence identity thereto; and wherein the VL comprises a complementarity determining region 1 (VL CDR1) comprising the amino acid sequence of SEQ ID NO: 9 or an amino acid sequence having at least 80% sequence identity thereto, a VL CDR2 comprising the amino acid sequence of SEQ ID NO: 10 or an amino acid sequence having at least 80% sequence identity thereto, and a VL CDR3 comprising the amino acid sequence of SEQ ID NO: 11 or an amino acid sequence having at least 80% sequence identity thereto, wherein the amino acid sequences of the CDR are based on IMGT numbering.

2. An antigen-binding molecule that specifically binds to an epitope of canine IL-31 comprising amino acid residues L29, Y32, Q33 and P40 of SEQ ID NO:1.

3. An antigen-binding molecule that competes for binding to canine IL-31 of SEQ ID NO:1 with the antigen-binding molecule of claim 1 or claim 2.

4. The antigen-binding molecule of any one of claims 1 to 3, wherein the VH comprises a VH CDR1 comprising the amino acid sequence of SEQ ID NO: 6, a VH CDR2 comprising the amino acid sequence of SEQ ID NO: 7 and a VH CDR3 comprising the amino acid sequence of SEQ ID NO: 8; and wherein the VL comprises a VL CDR1 comprising the amino acid sequence of SEQ ID NO: 9, a VL CDR2 comprising the amino acid sequence of SEQ ID NO: 10, and a VL CDR3 comprising the amino acid sequence of SEQ ID NO: 11.

5. The antigen-binding molecule of claim 4, wherein the VH comprises a VH CDR1 consisting of the amino acid sequence of SEQ ID NO: 6, a VH CDR2 consisting of the amino acid sequence of SEQ ID NO: 7 and a VH CDR3 consisting of the amino acid sequence of SEQ ID NO: 8; and wherein the VL comprises a VL CDR1 consisting of the amino acid sequence of SEQ ID NO: 9, a VL CDR2 consisting of the amino acid sequence of SEQ ID NO: 10, and a VL CDR3 consisting of the amino acid sequence of SEQ ID NO: 11.

6. The antigen-binding molecule of any one of claims 1 to 5, wherein the antigen-binding molecule does not compete for binding to IL-31 with (i) an IL-31-binding molecule that specifically binds to a region of IL-31 that corresponds to amino acid positions 13, 15, 20 and 26 of SEQ ID NO:1 or (ii) an IL-31 binding molecule that specifically binds to a region of IL-31 that corresponds to amino acid positions 76, 77, 80, 81, and 84 of SEQ ID NO:1.

7. The antigen-binding molecule of any one of claims 1 to 6, wherein the molecule is a caninized, felinized or equinized antigen-binding molecule.

8. The antigen-binding molecule of claim 7, wherein the molecule is a caninized antigen-binding molecule.

9. The antigen-binding molecule of claim 8, wherein the caninized antigen-binding molecule comprises:
(i) a heavy chain variable domain (VH) comprising:
(a) a heavy chain variable domain framework region 1 (VHFR1) amino acid sequence having at least 80% sequence identity to a VHFR1 amino acid sequence selected from the group consisting of SEQ ID NO: 18, 22, 26, 30 and 34,
(b) a VHFR2 amino acid sequence having at least 80% sequence identity to a VHFR2 amino acid sequence selected from the group consisting of SEQ ID NO: 19, 23, 27, 31 and 35,
(c) a VHFR3 amino acid sequence having at least 80% sequence identity to a VHFR3 amino acid sequence selected from the group consisting of SEQ ID NO: 20, 24, 27, 32 and 36, and
(d) a VHFR4 amino acid sequence having at least 80% sequence identity to a VHFR4 amino acid sequence selected from the group consisting of SEQ ID NO: 21, 25, 28, 33 and 37; and/or
(ii) a light chain variable domain (VL) comprising:
(e) a heavy chain variable domain framework region 1 (VLFR1) amino acid sequence having at least 80% sequence identity to a VLFR1 amino acid sequence selected from the group consisting of SEQ ID NO: 38, 42, 46 and 50,
(f) a VLFR2 amino acid sequence having at least 80% sequence identity to a VLFR2 amino acid sequence selected from the group consisting of SEQ ID NO, 39, 43, 47 and 51,
(g) a VLFR3 amino acid sequence having at least 80% sequence identity to a VLFR3 amino acid sequence selected from the group consisting of SEQ ID NO: 40, 44, 48 and 52, and
(h) a VLFR4 amino acid sequence having at least 80% sequence identity to a VHFR4 amino acid sequence selected from the group consisting of SEQ ID NO: 41, 45, 49 and 53.

10. The antigen-binding molecule of claim 8, wherein the caninized antigen-binding molecule comprises:
(i) a heavy chain variable domain (VH) comprising:
(i) a heavy chain variable domain framework region 1 (VHFR1) amino acid sequence having at least 80% sequence identity to a VHFR1 amino acid sequence of SEQ ID NO: 81 or SEQ ID NO:85,
(j) a VHFR2 amino acid sequence having at least 80% sequence identity to a VHFR2 amino acid sequence of SEQ ID NO: 82 or SEQ ID NO:86,
(k) a VHFR3 amino acid sequence having at least 80% sequence identity to a VHFR3 amino acid sequence of SEQ ID NO: 83 or SEQ ID NO:87, and
(l) a VHFR4 amino acid sequence having at least 80% sequence identity to a VHFR4 amino acid sequence of SEQ ID NO: 84 or SEQ ID NO:88; and/or
(ii) a light chain variable domain (VL) comprising:
(m) a heavy chain variable domain framework region 1 (VLFR1) amino acid sequence having at least 80% sequence identity to a VLFR1 amino acid sequence of SEQ ID NO: 89,
(n) a VLFR2 amino acid sequence having at least 80% sequence identity to a VLFR2 amino acid sequence of SEQ ID NO: 90,
(o) a VLFR3 amino acid sequence having at least 80% sequence identity to a VLFR3 amino acid sequence of SEQ ID NO: 91, and
(p) a VLFR4 amino acid sequence having at least 80% sequence identity to a VHFR4 amino acid sequence of SEQ ID NO: 92.

11. The antigen-binding molecule of claim 8, wherein the heavy chain variable domain (VH) comprises an amino acid sequence having at least 80% sequence identity to a VH amino acid sequence of SEQ ID NO: 99 or SEQ ID NO:100.

12. The antigen-binding molecule of claim 8, wherein the light chain variable domain (VL) comprises an amino acid sequence having at least 80% sequence identity to a VL amino acid sequence of SEQ ID NO: 101.

13. The antigen-binding molecule of claim 7, wherein the molecule is a felinized antigen-binding molecule.

14. The antigen-binding molecule of claim 13, wherein the felinized antigen-binding molecule comprises:
(i) a heavy chain variable domain (VH) comprising:
(a) a heavy chain variable domain framework region 1 (VHFR1) amino acid sequence having at least 80% sequence identity to a VHFR1 amino acid sequence of SEQ ID NO: 54,
(b) a VHFR2 amino acid sequence having at least 80% sequence identity to a VHFR2 amino acid of SEQ ID NO: 55,
(c) a VHFR3 amino acid sequence having at least 80% sequence identity to a VHFR3 amino acid sequence of SEQ ID NO: 56, and
(d) a VHFR4 amino acid sequence having at least 80% sequence identity to a VHFR4 amino acid sequence of SEQ ID NO: 57; and/or
(ii) a light chain variable domain (VL) comprising:
(e) a light chain variable domain framework region 1 (VLFR1) amino acid sequence having at least 80% sequence identity to a VLFR1 amino acid sequence of SEQ ID NO:58,
(f) a VLFR2 amino acid sequence having at least 80% sequence identity to a VLFR2 amino acid sequence of SEQ ID NO:59,
(g) a VLFR3 amino acid sequence having at least 80% sequence identity to a VLFR3 amino acid sequence of SEQ ID NO: 60, and
(h) a VLFR4 amino acid sequence having at least 80% sequence identity to a VHFR4 amino acid sequence of SEQ ID NO: 61.

15. The antigen-binding molecule of any one of the claims 1 to 14, wherein the antigen-binding molecule is an antibody or an IL-31-binding fragment thereof.

16. The antigen-binding molecule of claim 15, wherein the IL-31-binding fragment is selected from the group consisting of a Fab fragment, an scFab, an Fab′, a single chain variable fragment (scFv) and a one-armed antibody.

17. The antigen-binding molecule of any one of claims 1 to 16, wherein the antigen-binding molecule comprises a heavy chain and/or a light chain constant region of a canine, feline or equine immunoglobulin molecule.

18. The antigen-binding molecule of claim 17, wherein the immunoglobulin molecule is a feline immunoglobulin.

19. The antigen-binding molecule of claim 18, wherein the feline immunoglobulin molecule is selected from the group consisting of IgG1a, IgG1b and IgG2.

20. The antigen-binding molecule of claim 17, wherein the immunoglobulin molecule is a canine immunoglobulin molecule.

21. The antigen-binding molecule of claim 20, wherein the canine immunoglobulin molecule is selected from the group consisting of IgGA, IgGB, IgGC and IgGD.

22. The antigen-binding molecule of claim 21, wherein the canine immunoglobulin molecule is a canine IgGA.

23. The antigen-binding molecule of claim 22, wherein the canine immunoglobulin molecule comprises the amino acid sequence of SEQ ID NO:2, or an amino acid sequence having at least 80% sequence identity thereto.

24. The antigen-binding molecule of claim 22 or claim 23, wherein the antibody comprises a heavy chain comprising the amino acid sequence of SEQ ID NO:16 or an amino acid sequence having at least 80% sequence identity thereto.

25. The antigen-binding molecule of any one of claims 22 to 24, wherein the antibody comprises a light chain comprising the amino acid sequence of SEQ ID NO:17 or an amino acid sequence having at least 80% sequence identity thereto.

26. An isolated nucleic acid molecule comprising a nucleic acid sequence encoding the antigen-binding molecule of any one of claims 1 to 25.

27. An expression construct comprising a nucleic acid sequence of claim 26.

28. A host cell comprising the expression construct of claim 27.

29. A vector comprising a nucleic acid sequence encoding the antigen-binding molecule of any one of claims 1 to 25.

30. The vector of claim 29, wherein the vector is an AAV vector.

31. A pharmaceutical composition comprising the antigen-binding molecule of any one of claims 1 to 25, and a pharmaceutically acceptable carrier.

32. A kit comprising the antigen-binding molecule of any one of claims 1 to 25, the vector of claim 29 or claim 30, or the pharmaceutical composition of claim 31.

33. A method of treating or preventing a condition associated with increased expression and/or increased activity of IL-31, the method comprising administering to a subject in need thereof the antigen-binding molecule of any one of claims 1 to 25, the vector of claim 29 or claim 30, or the pharmaceutical composition of claim 31.

34. The method of claim 33, wherein the condition associated with increased expression and/or increased activity of IL-31 is selected from the group consisting of pruritis and dermatitis.

35. The method of claim 34, wherein the condition is atopic dermatitis.

36. The method of claim 34, wherein the condition is pruritis.

37. A method of treating or preventing a tumour induced to proliferate by IL-31 and conditions associated therewith, the method comprising administering to a subject in need thereof the antigen-binding molecule of any one of claims 1 to 25, the vector of claim 29 or claim 30, or the pharmaceutical composition of claim 31.

38. The method of any one of claims 33 to 37, wherein the subject is selected from the group consisting of a canine, a feline and an equine.

39. Use of the antigen-binding molecule of any one of claims 1 to 25 or the vector of claim 29 or claim 30, in the manufacture of a medicament for treating or preventing a condition associated with increased expression and/or increased activity of IL-31 in a subject in need thereof.

40. The use of claim 39, wherein the condition associated with increased expression and/or increased activity of IL-31 is selected from the group consisting of pruritis and dermatitis.

41. The use of claim 40, wherein the condition is atopic dermatitis.

42. The use of claim 40, wherein the condition is pruritis.

43. Use of the antigen-binding molecule of any one of claims 1 to 25 or the vector of claim 29 or claim 30, in the manufacture of a medicament for treating or preventing a tumour induced to proliferate by IL-31 and conditions associated therewith in a subject in need thereof.

44. The use of any one of claims 39 to 43, wherein the subject is selected from the group consisting of a canine, a feline and an equine.

45. The antigen-binding molecule of any one of claims 1 to 25, the vector of claim 29 or claim 30, or the pharmaceutical composition of claim 31 for use in the treatment or prevention of a condition associated with increased expression and/or increased activity of IL-31 in a subject in need thereof.

46. The antigen-binding molecule, the vector or the pharmaceutical composition for use according to claim 45, wherein the condition associated with increased expression and/or increased activity of IL-31 is selected from the group consisting of pruritis and dermatitis.

47. The antigen-binding molecule, the vector or the pharmaceutical composition for use according to claim 46, wherein the condition is atopic dermatitis.

48. The antigen-binding molecule, the vector or the pharmaceutical composition for use according to claim 46, wherein the condition is pruritis.

49. The antigen-binding molecule of any one of claims 1 to 25, the vector of claim 29 or claim 30, or the pharmaceutical composition of claim 31 for use in the treatment or prevention of a tumour induced to proliferate by IL-31 and conditions associated therewith in a subject in need thereof.

50. The antigen-binding molecule, the vector or the pharmaceutical composition for use according to any one of claims 45 to 49, wherein the subject is selected from the group consisting of a canine, a feline and an equine.

51. An immunogenic composition capable of raising an immune response to IL-31, wherein the composition comprises (i) an immunogen comprising a B cell epitope of IL-31 and (ii) a pharmaceutically acceptable carrier, wherein the B cell epitope comprises an amino acid sequence corresponding to amino acid positions 29-40 of SEQ ID NO:1, or an amino acid sequence that has at least 80% sequence identity thereto, and wherein the immunogen does not comprise the amino acid sequence of a native IL-31 molecule.

52. The immunogenic composition of claim 51, wherein the immunogen consists of an amino acid sequence corresponding to amino acid positions 29-40 of SEQ ID NO:1, or an amino acid sequence that has at least 80% sequence identity thereto.

53. The immunogenic composition of claim 51, wherein the immunogen consists of an amino acid sequence corresponding to amino acid positions 29-40 of SEQ ID NO:1.
